(19) United States
(12) Patent Application Publication
(10) Pub. No.: US 2023/0248044 A1
(43) Pub. Date: Aug. 10, 2023

(54) FOOD PRODUCT

(71) Applicant: GOLO LLC, Newark, DE (US)

(72) Inventor: Christopher Brian LUNDIN, Newark, DE (US)

(21) Appl. No.: 18/305,041

(22) Filed: Apr. 21, 2023

Related U.S. Application Data
(63) Continuation-in-part of application No. 17/409,183, filed on Aug. 23, 2021.
Application No. 17/409,183 is a continuation of application No. 16/265,000, filed on Feb. 1, 2019, now abandoned.
Application No. 16/265,000 is a continuation-in-part of application No. 14/816,343, filed on Aug. 3, 2015, now abandoned.

(30) Foreign Application Priority Data
Aug. 4, 2014 (ZA) 2014/04619

(51) Int. Cl. A23L 33/00

(52) U.S. Cl. CPC A23L 33/30; A23L 33/22

(57) ABSTRACT

This application describes a food product that can be packaged from a food composition. The food product can be used in a weight management program. The food composition contains protein, including both plant-based proteins and/or animal-based proteins, plant-based complex or starch carbohydrates, fats, vegetables, natural herbs, and preservative mixture in a mixture that includes fiber and essential nutrients that can comprise a complete meal for a consumer. Other embodiments are described.

### CROSS REFERENCE TO RELATED APPLICATIONS

This application is a continuation-in-part of U.S. patent application Ser. No. 17/409,183, filed Aug. 23, 2021, which is a continuation of U.S. patent application Ser. No. 16/265,000, filed on Feb. 1, 2019, titled, “Food Product”, which is a continuation-in-part of U.S. patent application Ser. No. 14/816,343, filed on Aug. 3, 2015, titled, “Food Product”, which claim priority to South Africa Application No. ZA20140004619, filed Aug. 4, 2014, each of which is herein incorporated by reference in their entirety.

### BACKGROUND OF THE INVENTION

This invention relates to the management of an individual's weight in a nutritionally sound manner.

### Unhealthy Habits

Busy lifestyles can make it difficult for many consumers to prepare fresh wholefood for themselves and their families.

Food marketers have responded to the opportunity with the development and supply of highly processed convenient foods such as ready to eat fast food, frozen dinners and meals, protein and meal replacement bars, shakes, snacks, chips, cookies, candy bars and energy drinks.

To cut costs, improve yields and extend the shelf life of processed foods, animals are fed antibiotics and growth hormones; crops are genetically modified; high fructose corn syrup, additives, preservatives and dyes are added to food products; and protein replacement foods, such as soy, whey and manipulated animal proteins, are used.

These highly processed foods are marketed as healthy and convenient substitutes for wholefoods. Many consumers, instead of buying fresh wholefoods, cooking balanced meals and preparing wholefood snacks, have gravitated to convenience foods.

Convenience foods can have a negative effect on diet-induced thermogenesis and cause insulin spikes resulting in an increase in obesity and obesity-related conditions, such as insulin resistance, and diseases including heart disease, type-2 diabetes and some physical and neurological disorders.

Insulin resistance has the effect of working against weight loss, and facilitating weight gain.

### Pseudo Healthy Foods

As obesity levels increase, more aggressive measures are adopted in order to lose weight. Food and drink manufacturers have taken advantage of this situation by developing and marketing low fat and low calorie versions of convenience foods or low fat replacement meals, shakes or beverages and marketing them as “healthier” than the high calorie counterparts. These products frequently contain components of low nutritional value, such as artificial sugars, to reduce their calorie content. Often, additives, flavorings and preservatives are added to extend shelf life and improve taste.

The replacement meals normally contain high levels of sugars and are not complete, balanced meals. The high sugar levels can cause insulin spikes which promote the development of insulin resistance. This type of meal generally ranges between 150 and 250 calories with an average of around 200 calories. These meals on their own, based on three meals a day, do not meet recognized safe minimum caloric requirements (e.g. 1200 calories). Some diet companies recommend an intake of less than 800 calories per day—an unsafe level which is likely to promote muscle loss and dehydration, resulting in false weight loss, and hunger pangs or cravings which can cause binge eating. Considering their low calorie content, these meals are expensive and offer little value for money.

### Healthy Lifestyles

Despite the market being flooded with highly processed foods and food alternatives, there are consumers who wish to live healthy lifestyles and to manage their weights by consuming wholefoods. However, it is often challenging for a consumer to buy wholefood ingredients and then to prepare a balanced meal, while tracking the ratios of the essential nutrients present in the ingredients used to prepare the meal. Ideally, a balanced meal should include ingredients selected from foods which are healthy sources of fiber and the three or four essential nutrients, for example proteins, carbohydrates, vegetables, and fats. It has been shown that the nutrients should be present in specific ratios to stabilize and optimize insulin levels, and food ingredients should be selected keeping these ratios in mind.

The sources of the nutrients may influence the effect of a meal on a consumer's metabolism. A specific example is the source of carbohydrates. Both starch and vegetables are carbohydrate-rich; however the metabolic effects of vegetables and starch are not the same.

By relying only on information which is provided on labels, it is daunting and time consuming for a consumer to count and accumulate a daily caloric target, made up of foods selected from ingredients which, in combination, have the correct nutrient ratio. Attempting a similar exercise with fresh foods that have no packaging or labelling is extremely difficult.

To make life simpler, many consumers buy readymade meals, marketed as “balanced”, such as frozen dinners. Similar to the convenience foods, these ready-made meals are often manufactured using highly processed ingredients, additives, dyes and artificially manufactured or processed proteins, which cannot replace fresh wholefood.

Another problem faced by consumers wishing to live healthy lifestyles is confusing labelling. The USDA allows an error of flexibility on food labelling of up to 20% (e.g. calories could be up to 20% higher or lower than actually claimed on a label). Food manufacturers often refer to a recommended daily allowance (RDA) in a misleading manner, indicating, for example, that a frozen dinner's carbohydrate content is low and the protein content is high, relative to the RDA, leaving the consumer under the impression that the meal is healthy, when, in fact, it may have a high fat, preservative, sodium or additive content. The misconception is normally caused by referring to nutrient content on a label in terms of percentages. A relatively low amount of protein could be reflected in a high percentage value, due to the low RDA of protein. Similarly, a high amount of carbohydrates could have a low percentage value. This misconception could lead a consumer to interpret the label incorrectly, inferring that the meal contains “less bad and more good”.

Another misleading concept regarding healthy lifestyles is the six meals a day philosophy. Many trainers, dieticians and diet companies recommend that dieters should eat every 2 to 3 hours to maintain sugar levels. This normally equates to 6 to 8 mini-meals per day. This philosophy is based on the following reasoning:

1. It satisfies an urge to eat excessively by providing smaller, more regular, mini-meals; and

2. It manages blood sugar levels throughout the day, decreasing spikes in insulin levels

Problems with the philosophy are the following;

1. As glucose in the bloodstream is constantly replenished by the consumed meals, the body does not release stored fat as glucose. Insulin is also constantly present in the bloodstream, due to the body's insulin response to glucose levels;

2. Following a traditional calorie restrictive diet of 1200 calories results in an average mini-meal of only 150 to 200 calories (8 to 6 meals). Creating a mini-meal that has all 3 or 4 essential nutrients plus fiber, in the right proportions, is extremely difficult and time consuming and inevitably results in a consumer dropping key nutrients, for example by only eating a fruit or easy-on-the-go snacks like protein and fiber bars, which are all high in sugars and other processed ingredients. This causes a consumer to loose muscle mass and does not create a sustainable weight management system.

### The USDA-FDA Established Recommended Daily Allowance

The RDA nutritional content, as established by the USDA-FDA for a 2000 calorie diet, is reflected in Table 1.

TABLE 1

| Food Group | Weight in grams | Percentage content |
|---|---|---|
| Total Carbohydrates | 300 | 68% |
| Protein | 50 | 11% |
| Fiber | 25 | 6% |
| Fat | 65 | 15% |

From Table 1 it can be seen that if a consumer were to adopt an RDA approach, he would be consuming significantly more carbohydrates and fats than fiber and protein to achieve the nutritional recommendation. This disproportionate ratio is not conducive to good health nor to insulin management.

Criticisms against the RDA approach are that it fails to account for individual differences, and it fails to address disease prevention and optimal nourishment. As such, an RDA basis may be considered as only being suitable for an individual who is healthy and is at an optimum weight.

Another shortcoming of an RDA guideline is that it does not place emphasis on consuming balanced meals which include ingredients which contain fiber and the three nutrients. Nor does the RDA approach emphasize that the nutrients should come from healthy sources. Meals that are not balanced and that do not contain nutrients from a variety of sources do not manage insulin levels effectively, and cause insulin spikes. For example, obtaining carbohydrates from only one source, such as starch, is not healthy—it is important for consumers to realize that a large portion of their daily carbohydrate allowance should be obtained from vegetable sources.

This specification is to be read in conjunction with the disclosure in the applicant's international application No. PCT/IB2014/000137 (“the earlier specification”) the content of which is hereby wholly incorporated into this specification.

The present invention is concerned with providing a food product to simplify the effective implementation and conduct of a weight management program which could include weight loss, weight gain, or weight management, goals, according to a consumer's requirements.

### SUMMARY OF THE INVENTION

The invention provides a bulk food product for use in a weight management program, which product is manufactured at a location remote from a consumer, with sufficient content for at least one portion of a defined size which constitutes a meal, the content being made up of ingredients of essential nutrients including protein, carbohydrate, vegetables, and fat, wherein the essential nutrients may be present in the following proportion to stabilize insulin levels, on a per unit mass basis;

1. a protein source in the amount of 26 to 40 mass units

2. a fat source in the amount of 7 to 16 mass units;

3. a carbohydrate source in the amount of 15 to 20 mass units; and

4. a vegetable source in the amount of 20 to 30 mass units.

The invention provides a bulk food product for use in a weight management program, which product is manufactured at a location remote from a consumer, with sufficient content for at least one portion of a defined size which constitutes a meal, the content being made up of ingredients which can be healthy sources of fiber, and of essential nutrients including protein, carbohydrate, vegetables, and fat, wherein the fiber and essential nutrients may be present in the following proportion to stabilize insulin levels, on a per unit mass basis;

1. a protein source in the amount of 26 to 40 mass units

2. a fat source in the amount of 7 to 16 mass units;

3. a carbohydrate source in the amount of 15 to 20 mass units;

4. a vegetable source in the amount of 20 to 30 mass units; and

5. a fiber source in the amount of 11 to 21 mass units.

“Weight management”, in terms of this specification, refers to weight loss, weight gain or weight maintenance, according to the circumstances.

As used herein, the term “about” means plus or minus 10% of the numerical value of the number with which it is being used. Therefore, about 50% means in the range of 45% to 55%.

The bulk food product preferably comprises a plurality of complete balanced meal portions, each portion having the correct ratio of essential nutrients and fiber.

In some embodiments, the bulk food product may comprise essential nutrients wherein the essential nutrients may comprise a protein, a carbohydrate, a vegetable, or a fat.

The food product may be used to implement any type of balanced diet and can be used for weight loss, weight maintenance or weight gain purposes, or as a snack or meal substitute.

The food product may be safely used to implement a calorie restricted diet for weight loss, by reducing the amount of the food product which is consumed per meal.

The food product may be used, for example by adolescents or athletes, to gain weight healthily by increasing the size of the portion of the food product, i.e. the amount, consumed per meal. Additional portions may also be consumed as snacks between meals.

A side dish, such as an egg or a salad, may be added as required by the consumer. For example, an athlete/bodybuilder may wish to add an extra protein source in the form of an egg etc. In some embodiments, a side dish may comprise an essential nutrient, for example a protein source, a carbohydrate source, a vegetable source, or a fat source.

[While a meal portion of the bulk food product is flexible, a baseline serving has been established as between 400 g to 525 g of the bulk food product. This is approximately equal to 400 to 525 calories. Preferably, the baseline serving is about 450 g of the food product which provides approximately 450 calories.

In some embodiments, each baseline serving may include ingredients, which may provide the essential nutrients, in the following quantities:

1. a protein source in the amount of about 26 g to about 40 g;

2. a fat source in the amount of about 7 g to about 16 g;

3. a carbohydrate source in the amount of about 15 g to about 20 g;

4. a vegetable source in the amount of about 20 g to about 30 g;

5. optionally, a proprietary food blend in the amount of about 1 g to about 10 g;

6. optionally, a proprietary natural preservative blend in the amount of about 1 g to about 3 g.

In some embodiments, each baseline serving may include ingredients, which may provide the fiber and the essential nutrients, in the following quantities:

1. a protein source in the amount of about 26 g to about 40 g;

2. a fat source in the amount of about 7 g to about 16 g;

3. a carbohydrate source in the amount of about 15 g to about 20 g;

4. a vegetable source in the amount of about 20 g to about 30 g;

5. a fiber source in the amount of about 11 g to about 21 g;

6. optionally, a proprietary food blend in the amount of about 1 g to about 10 g;

7. optionally, a proprietary natural preservative blend in the amount of about 1 g to about 3 g.

In some embodiments, each baseline serving may include ingredients, which may provide the essential nutrients, in the following quantities:

1. a protein source in the amount of about 14 g to about 128 g;

2. a fat source in the amount of about 4 g to about 42 g;

3. a carbohydrate source in the amount of about 25 g to about 80 g;

4. a vegetable source in the amount of about 40 g to about 128 g;

5. optionally, a proprietary food blend in the amount of about 1 g to about 10 g;

6. optionally, a proprietary natural preservative blend in the amount of about 1 g to about 3 g.

In some embodiments, each baseline serving may include ingredients, which may provide the fiber and the essential nutrients, in the following quantities:

1. a protein source in the amount of about 14 g to about 128 g;

2. a fat source in the amount of about 4 g to about 42 g;

3. a carbohydrate source in the amount of about 25 g to about 80 g;

4. a vegetable source in the amount of about 40 g to about 128 g;

5. a fiber source in the amount of about 1 g to about 50 g;

6. optionally, a proprietary food blend in the amount of about 1 g to about 10 g;

7. optionally, a proprietary natural preservative blend in the amount of about 1 g to about 3 g.

In some embodiments, each baseline serving may include ingredients, which may provide the essential nutrients, in the following quantities:

1. a protein source in the amount of about 28 g to about 256 g;

2. a fat source in the amount of about 8 g to about 84 g;

3. a carbohydrate source in the amount of about 50 g to about 160 g;

4. a vegetable source in the amount of about 80 g to about 256 g;

5. optionally, a proprietary food blend in the amount of about 2 g to about 20 g;

6. optionally, a proprietary natural preservative blend in the amount of about 2 g to about 6 g.

In some embodiments, each baseline serving may include ingredients, which may provide the fiber and the essential nutrients, in the following quantities:

1. a protein source in the amount of about 28 g to about 256 g;

2. a fat source in the amount of about 8 g to about 84 g;

3. a carbohydrate source in the amount of about 50 g to about 160 g;

4. a vegetable source in the amount of about 80 g to about 256 g;

5. a fiber source in the amount of about 2 g to about 100 g;

6. optionally, a proprietary food blend in the amount of about 2 g to about 20 g;

7. optionally, a proprietary natural preservative blend in the amount of about 2 g to about 6 g.

In some embodiments, each baseline serving may include ingredients, which may provide the essential nutrients, in the following quantities:

1. a protein source in the amount of about 14 g to about 128 g;

2. a fat source in the amount of about 4 g to about 42 g;

3. a carbohydrate source in the amount of about 25 g to about 80 g;

4. a vegetable source in the amount of about 40 g to about 128 g;

5. optionally, a proprietary food blend in the amount of about 1 g to about 10 g;

6. optionally, a proprietary natural preservative blend in the amount of about 1 g to about 3 g.

In some embodiments, each baseline serving may include ingredients, which may provide the fiber and the essential nutrients, in the following quantities:

1. a protein source in the amount of about 14 g to about 128 g;

2. a fat source in the amount of about 4 g to about 42 g;

3. a carbohydrate source in the amount of about 25 g to about 80 g;

4. a vegetable source in the amount of about 40 g to about 128 g;

5. a fiber source in the amount of about 1 g to about 50 g;

6. optionally, a proprietary food blend in the amount of about 1 g to about 10 g;

7. optionally, a proprietary natural preservative blend in the amount of about 1 g to about 3 g.

In some embodiments, each baseline serving may include ingredients, which may provide the essential nutrients, in the following quantities:

1. a protein source in the amount of about 28 g to about 256 g;

2. a fat source in the amount of about 8 g to about 84 g;

3. a carbohydrate source in the amount of about 50 g to about 160 g;

4. a vegetable source in the amount of about 80 g to about 256 g;

5. optionally, a proprietary food blend in the amount of about 2 g to about 20 g;

6. optionally, a proprietary natural preservative blend about 2 g to about 6 g.

In some embodiments, each baseline serving may include ingredients, which may provide the fiber and the essential nutrients, in the following quantities:

1. a protein source in the amount of about 28 g to about 256 g;

2. a fat source in the amount of about 8 g to about 84 g;

3. a carbohydrate source in the amount of about 50 g to about 160 g;

4. a vegetable source in the amount of about 80 g to about 256 g;

5. a fiber source in the amount of about 2 g to about 100 g;

6. optionally, a proprietary food blend in the amount of about 2 g to about 20 g;

7. optionally, a proprietary natural preservative blend in the amount of about 2 g to about 6 g.

In some embodiments, each baseline serving may include ingredients in the following quantities:

1. a protein source in the amount of about 14 g to about 128 g;

2. a fat source in the amount of about 4 g to about 42 g;

3. a carbohydrate source in the amount of about 25 g to about 80 g;

4. a vegetable source in the amount of about 40 g to about 128 g.

In some embodiments, each baseline serving may include ingredients in the following quantities:

1. a protein source in the amount of about 28 g to about 256 g;

2. a fat source in the amount of about 4 g to about 42 g;

3. a carbohydrate in the amount of about 50 g to about 160 g;

4. a vegetable source in the amount of about 40 g to about 128 g.

In some embodiments, each baseline serving may include ingredients in the following quantities:

1. a protein source in the amount of about 28 g to about 256 g;

2. a fat source in the amount of about 4 g to about 42 g;

3. a carbohydrate source in the amount of about 50 g to about 160 g;

4. optionally, a vegetable source in the amount of about 40 g to about 128 g.

In some embodiments, each baseline serving may include ingredients in the following quantities:

1. a protein source in the amount of about 28 g to about 256 g;

2. a fat source in the amount of about 4 g to about 42 g;

3. a carbohydrate source in the amount of about 25 g to about 80 g;

4. a vegetable source in the amount of about 80 g to about 256 g.

In some embodiments, each baseline serving may include ingredients in the following quantities:

1. a protein source in the amount of about 14 g to about 128 g;

2. a fat source in the amount of about 4 g to about 42 g;

3. a carbohydrate source in the amount of about 25 g to about 80 g;

4. a vegetable source in the amount of about 80 g to about 256 g.

In some embodiments, each baseline serving may include ingredients in the following quantities:

1. a protein source in the amount of about 28 g to about 256 g;

2. a fat source in the amount of about 8 g to about 84 g;

3. a carbohydrate source in the amount of about 50 g to about 160 g;

4. a vegetable source in the amount of about 80 g to about 256 g.

The protein may be obtained from food sources known to be high in protein. Specifically, the protein source may be selected from lean meat, such as chicken without skin, beef, pork and fish. Alternatively, the protein source may be selected from a variety of beans to create a vegetarian friendly bulk food product. In some embodiments, the protein source may include, but not limited to, beef, bison, buffalo, chicken, duck, goose, hen, lamb, pork, quail, turkey, veal, venison, crab, bass, catfish, clams, cod, flounder, grouper, halibut, haddock, lobster, mussels, oysters, rockfish, salmon, scallops, shrimp, snapper, swordfish, trout, tuna, cheese (all natural), cottage cheese, cream cheese, eggs, milk (whole), yogurt (plain), raw nuts (all varieties), seeds (pumpkin sunflower), seeds (hemp, chia, etc.), tempeh, or tofu.

In some embodiments, the protein may have a serving size. In some embodiments, the protein serving size is in a range from about 14 g to about 128 g. In some embodiments, the protein serving size is in a range from about 28 g to about 256 g. In some embodiments, the protein serving size is in a range from about 50 g to about 85 g. In some embodiments, the protein serving size is in a range from about 32 g to about 128 g. In some embodiments, the protein serving size is in a range from about 14 g to about 42 g. In some embodiments, the protein serving size is 50 g. In some embodiments, the protein serving size is 85 g. In some embodiments, the protein serving size is in a range from about 1 ounce to about 3 ounces. In some embodiments, the protein serving size is in a range from about 2 ounces to about 6 ounces. In some embodiments, the protein serving size is in a range from about 2 ounces to about 3 ounces. In some embodiments, the protein serving size is in a range from about 1 ounce to about 2 ounces. In some embodiments, the protein serving size is 3 ounces. In some embodiments, the protein serving size is 2 ounces. In some embodiments, the protein serving size is in a range from about ¼ cup to about 1 cup. In some embodiments, the protein serving size is in a range from about ½ cups to about 2 cups. In some embodiments, the protein serving size is in a range from about ½ cup to about 1 cup. In some embodiments, the protein serving size is about ½ cup. In some embodiments, the protein serving size is in a range from about 1 tablespoon to about 3 tablespoons. In some embodiments, the protein serving size is in a range from about 2 tablespoons to about 6 tablespoons. In some embodiments, the protein serving size is in a range from about 2 tablespoons to about 3 tablespoons. In some embodiments, the protein serving size is 2 tablespoons. In some embodiments, the protein serving size is 3 tablespoons. In some embodiments, the protein serving size is 1 egg.

The vegetable source may be obtained from any vegetable source including, but not limited to, carrot, spinach and other vegetables. In some embodiments, the vegetable can be a non-starchy vegetable (http://www.diabetes.org/food-and-fitness/food/what-can-i-eat/making-heal-thy-food-choices/non-starchy-vegetables.html). In some embodiments, the vegetable source may include, but not limited to, alfalfa sprouts, artichokes, arugula, asparagus, bean sprouts, beets, bok choy, broccoli (all varieties), Brussels sprouts, cabbage (green), cabbage (red), carrots, cauliflower, celery, collard greens, cucumber, eggplant, fennel, green beans, kale, leeks, lettuce, mushrooms, okra, onion, parsnips, peppers (green), peppers (all other), pumpkin, radish, sauerkraut, spinach, squash (all varieties), Swiss chard, snow peas, turnips, zucchini, avocado, tomatoes, tomato sauce, olives, corn, or peas.

In some embodiments, the vegetable source may have a serving size. In some embodiments, the vegetable serving size is in a range from about 40 g to about 128 g. In some embodiments, the vegetable serving size is in a range from about 80 g to about 256 g. In some embodiments, the vegetable serving size is in a range from about 64 g to about 128 g. In some embodiments, the vegetable serving size is about 64 g. In some embodiments, the vegetable serving size is about 128 g. In some embodiments, the vegetable serving size is in a range from about ½ cup to about 1 cup. In some embodiments, the vegetable serving size is in a range from about 1 cup to about 2 cups. In some embodiments, the vegetable serving size is about ½ cup. In some embodiments, the vegetable serving size is about 1 cup. In some embodiments, the vegetable serving size is 8 olives. In some embodiments, the vegetable serving size is 16 olives.

The carbohydrate source may be obtained from any starch source including, but not limited to, potatoes, rice and yams (sweet potato). In some embodiments, the carbohydrate source may include, but not limited to, apple, apricot, banana, blackberries, blueberries, cantaloupe, cherries, grapefruit, grapes, guava, honeydew, kiwi, mango, nectarine, orange, papaya, peach, pear, pineapple, plantain, plum, raspberries, strawberries, watermelon, dried fruit, potatoes, sweet potatoes, yams, amaranth, barley, bread, brown rice, corn tortilla, wheat tortilla, couscous, farro, millet, spelt, oatmeal, pasta, quinoa, jicama, whole wheat, coconut flour, beans (all varieties), edamame, hummus, lentils, or lima beans.

In some embodiments, the carbohydrate source may have a serving size. In some embodiments, the carbohydrate serving size is in a range from about 25 g to about 80 g. In some embodiments, the carbohydrate serving size is in a range from about 50 g to about 160 g. In some embodiments, the carbohydrate serving size is in a range from about 32 g to about 64 g. In some embodiments, the carbohydrate serving size is 32 g. In some embodiments, the carbohydrate serving size is 64 g. In some embodiments, the carbohydrate serving size is in a range from about ¼ cup to about ½ cup. In some embodiments, the carbohydrate serving size is in a range from about ½ cup to about 1 cup. In some embodiments, the carbohydrate serving size is about ¼ cup. In some embodiments, the carbohydrate serving size is about ½ cup. In some embodiments, the carbohydrate serving size is 1 corn or wheat tortilla. In some embodiments, the carbohydrate serving size is 1 slice of bread.

The fat source may be obtained from any fat source including, but not limited to, butter, coconut oil, oils (olive, walnut, sesame, or avocado), homemade dressing, or unsweetened coconut flakes.

In some embodiments, the fat may have a serving size. In some embodiments, the fat serving size is in a range from about 4 g to about 42 g. In some embodiments, the fat serving size is in a range from about 14 g to about 42 g. In some embodiments, the fat serving size is in a range from about 1 teaspoon to about 2 teaspoons. In some embodiments, the fat serving size is about 1 teaspoon. In some embodiments, the fat serving size is about 2 teaspoons. In some embodiments, the fat serving size is in a range from about 1 tablespoon to about 3 tablespoons. In some embodiments, the fat serving size is in a range from about 1 tablespoon to about 2 tablespoons. In some embodiments, the fat serving size is in a range from about 2 tablespoons to about 3 tablespoons. In some embodiments, the fat serving size is about 1 tablespoon. In some embodiments, the fat serving size is about 2 tablespoons. In some embodiments, the fat serving size is about 3 tablespoons.

The proprietary food blend includes a selection of natural plant based foods, such as wheat grass, flax, wheat germ and other ingredients high in nutrition and fiber. The blend may also include enzymes and probiotics that are conducive to health.

The natural preservative blend may include natural ingredients which preserve food and provide flavour such as sea salt, lemon, rosemary oil, garlic and other natural flavourings.

The ingredients may be selected to create a variety of different bulk food products suitable to different tastes e.g. suitable ingredients can be selected to produce a sweet food product, a savoury food product, a vegetarian food product, etc.

The size of a serving is preferably adequate to provide sufficient energy to support the caloric requirements of a consumer for about 3 to 5 hours depending on the consumer's energy expenditure or objective.

A serving of the food product may be diluted to create a stew or a soup. The food product may also be customized for an individual consumer's taste by adding spices and natural flavorings.

The bulk food product may be sold via an online system, using appropriate software, and may be delivered to a consumer's specified address.

The bulk food product, or portions thereof, may also be delivered to retail outlets or restaurants.

Although the food product may be provided as a single portion it is preferably delivered in bulk form, frozen or refrigerated.

The various food ingredients of the bulk food product are prepared separately under controlled conditions, at a food preparation facility remote from a consumer, and are combined, mixed or blended to form the bulk food product which is then packaged into one or more appropriate containers for shipment or transport.

In one example, the bulk food product is used to implement a weight management program in terms of which a consumer establishes his/her daily base food allowance or requirements, in a defined unit system which contains an index of food values and selects food for consumption based at least on food index values in the system.

The number of units of the food consumed each day by the consumer should be within 90 to 110% of the consumer's daily base food allowance expressed in the units.

The food value index includes defined portions of foods, categorized in terms of their respective content of essential nutrients. A food value, in the units, is assigned to each food portion. The food value is determined using certain parameters including calories, protein content, carbohydrate content, fiber content, fat content and processed content.

The daily base food allowance may be established using parameters that pertain to each individual consumer including gender, age, height, weight, waist circumference and life activity. Each parameter has a set of categories associated with a predetermined value. For example a gender category includes male and female classes; an age category may be divided into ranges for example from 20 to 30 years, from 30 to 40 years, etc.; a life activity category may, for example, allow for a sedentary, inactive, moderately active, active or very active, lifestyle. Parameters relating to height and weight are used in combination to determine a body mass index (BMI), a concept known in the art. A BMI range is also divided into classes, e.g. from 18 to 24, from 24 to 30, etc.

To establish the daily base food allowance, a consumer accumulates the values from each category that are relevant to his/her measurements—the total constitutes the daily base food allowance.

An exercise index may be established wherein values (in the aforementioned units) are assigned to particular exercises of defined durations. A consumer may increase his daily base food allowance by adding the unit values of the exercises to his initial daily base food allowance. This, in turn, will allow the consumer to consume a correspondingly greater quantity of the food product.

The bulk food product comprises a plurality of complete balanced meal portions. Each portion is associated with a food value (in the units) that is calculated based on the relevant food values linked to the ingredients contained in the product, making it easy for the consumer to keep track of the number of units that have been consumed and to correlate this consumption with his daily base food allowance, typically of the order of 450 units to 500 units depending on activity. When consuming a meal/serving of the food product, the consumer is assured that the meal is balanced and contains ingredients selected from all varieties of foods, categorised in terms of their fiber and essential nutrient content, ensuring that the essential nutrients are present in a ratio which is optimal for stabilising insulin resistance.

An additional portion of a complete meal or additional ingredients or a side dish e.g. an egg or a salad, may be added to make up a shortfall between the food index points of a complete meal and the daily base food allowance of the consumer.

The consumer can choose when to eat all or a part of his/her daily base food allowance. The responsibility or discretion placed on a consumer to select ingredients is removed. Psychologically, this has a substantial benefit in that the consumer is relieved of the flexibility of choice and, when eating a complete meal product is inherently assured of consuming the appropriate number of food units, containing fiber and the three essential nutrients in the correct combinations and proportions.

### DESCRIPTION OF FIGURES

FIG. 1 illustrates change in average body weight (lb) and waist circumference after 7 days, 30 days and 90 days.

FIG. 2 illustrates change in average body weight (lb) and body mass index (BMI) after 26 weeks.

FIG. 3 illustrates change in average body weight and BMI after 26 weeks as a percentage of baseline values.

FIG. 4 illustrates change in average fasting blood glucose (FBG) and hemoglobin Ale from baseline to 26 weeks (n=35).

FIG. 5 illustrates change in average blood pressure from baseline to 26 weeks (n=35).

FIG. 6 illustrates change in average visceral fat (%) over 26 weeks (p=0.005) and over 39-55 weeks (p<0.00001) (n=35)).

### DETAILED DESCRIPTION

The following description refers to Tables 2 to 6, attached hereto.

The invention provides a bulk food product for use in a weight management program, made up from a mixture of primary ingredients selected from whole foods containing fiber and the three or four essential nutrients, i.e. proteins, carbohydrates, fat, or vegetables. The food product may further comprise a proprietary food mixture; and a blend of natural preservatives, combined in predetermined proportions shown to stabilize and optimize insulin levels.

The food product is used to implement a diet plan for maintaining body weight, losing weight or gaining weight in a healthy and nutritious way.

The ingredients are cooked separately, to optimize flavour and nutritional value, and the cooked ingredients are then combined or mixed to form the bulk food product.

The mixture includes sufficient content for multiple balanced meals or meal portions, where each meal has the bulk food product, provided to a consumer, would include sufficient content for 1 to 24 meals.

460 g of the mixture comprises a single portion or meal which provides approximately 460 calories. If necessary (depending on a consumer's goal e.g. weight loss, weight gain or weight management) additional ingredients or a side dish, such as an egg or a salad, can be included with a meal.

Optionally, the food product can be consumed in smaller portions, as snacks, by reducing the amount consumed at a time to, say, a third of a portion/half a cup. A quantity of the food product can also be diluted to create a stew or soup, to produce a nutritious, cost-effective meal.

An example of amounts of ingredients included per meal, is as follows;

1. a protein source in the amount of 26 g to 40 g

2. a fat source in the amount of 7 g to 16 g;

3. a carbohydrate source in the amount of 15 g to 20 g;

4. a vegetable source in the amount of 20 g to 30 g;

5. optionally, a proprietary food blend in the amount of 1 to 10 g;

6. optionally, a proprietary natural preservative blend in the amount of 1 to 3 g.

An example of amounts of ingredients included per meal, is as follows;

1. a protein source in the amount of 26 g to 40 g

2. a fat source in the amount of 7 g to 16 g;

3. a carbohydrate source in the amount of 15 g to 20 g;

4. a vegetable source in the amount of 20 g to 30 g;

5. a fiber source in the amount of 11 g to 21 g;

6. optionally, a proprietary food blend in the amount of 1 to 10 g;

7. optionally, a proprietary natural preservative blend in the amount of 1 to 3 g.

In some embodiments, each baseline serving may include ingredients, which may provide the essential nutrients, in the following quantities:

1. a protein source in the amount of 14 g to 128 g;

2. a fat source in the amount of 4 g to 42 g;

3. a carbohydrate source in the amount of 25 g to 80 g;

4. a vegetable source in the amount of 40 g to 128 g;

5. optionally, a proprietary food blend in the amount of 1 g to 10 g;

6. optionally, a proprietary natural preservative blend in the amount of 1 g to 3 g.

In some embodiments, each baseline serving may include ingredients, which may provide the fiber and the essential nutrients, in the following quantities:

1. a protein source in the amount of 14 g to 128 g;

2. a fat source in the amount of 4 g to 42 g;

3. a carbohydrate source in the amount of 25 g to 80 g;

4. a vegetable source in the amount of 40 g to 128 g;

5. a fiber source in the amount of 1 g to 50 g;

6. optionally, a proprietary food blend in the amount of 1 g to 10 g;

7. optionally, a proprietary natural preservative blend in the amount of 1 g to 3 g.

In some embodiments, each baseline serving may include ingredients, which may provide the essential nutrients, in the following quantities:

1. a protein source in the amount of 28 g to 256 g;

2. a fat source in the amount of 8 g to 84 g;

3. a carbohydrate source in the amount of 50 g to 160 g;

4. a vegetable source in the amount of 80 g to 256 g;

5. optionally, a proprietary food blend in the amount of 2 g to 20 g;

6. optionally, a proprietary natural preservative blend in the amount of 2 g to 6 g.

In some embodiments, each baseline serving may include ingredients, which may provide the fiber and the essential nutrients, in the following quantities:

1. a protein source in the amount of 28 g to 256 g;

2. a fat source in the amount of 8 g to 84 g;

3. a carbohydrate source in the amount of 50 g to 160 g;

4. a vegetable source in the amount of 80 g to 256 g;

5. a fiber source in the amount of 2 g to 100 g;

6. optionally, a proprietary food blend in the amount of 2 g to 20 g;

7. optionally, a proprietary natural preservative blend in the amount of 2 g to 6 g.

In some embodiments, each baseline serving may include ingredients, which may provide the essential nutrients, in the following quantities:

1. a protein source in the amount of 14 g to 128 g;

2. a fat source in the amount of 4 g to 42 g;

3. a carbohydrate source in the amount of 25 g to 80 g;

4. a vegetable source in the amount of 40 g to 128 g;

5. optionally, a proprietary food blend in the amount of 1 g to 10 g;

6. optionally, a proprietary natural preservative blend in the amount of 1 g to 3 g.

In some embodiments, each baseline serving may include ingredients, which may provide the fiber and the essential nutrients, in the following quantities:

1. a protein source in the amount of 14 g to 128 g;

2. a fat source in the amount of 4 g to 42 g;

3. a carbohydrate source in the amount of 25 g to 80 g;

4. a vegetable source in the amount of 40 g to 128 g;

5. a fiber source in the amount of 1 g to 50 g;

6. optionally, a proprietary food blend in the amount of 1 g to 10 g;

7. optionally, a proprietary natural preservative blend in the amount of 1 g to 3 g.

In some embodiments, each baseline serving may include ingredients, which may provide the essential nutrients, in the following quantities:

1. a protein source in the amount of 28 g to 256 g;

2. a fat source in the amount of 8 g to 84 g;

3. a carbohydrate source in the amount of 50 g to 160 g;

4. a vegetable source in the amount of 80 g to 256 g;

5. optionally, a proprietary food blend in the amount of 2 g to 20 g;

6. optionally, a proprietary natural preservative blend in the amount of 2 g to 6 g.

In some embodiments, each baseline serving may include ingredients, which may provide the fiber and the essential nutrients, in the following quantities:

1. a protein source in the amount of 28 g to 256 g;

2. a fat source in the amount of 8 g to 84 g;

3. a carbohydrate source in the amount of 50 g to 160 g;

4. a vegetable source in the amount of 80 g to 256 g;

5. a fiber source in the amount of 2 g to 100 g;

6. optionally, a proprietary food blend in the amount of 2 g to 20 g;

7. optionally, a proprietary natural preservative blend in the amount of 2 g to 6 g.

In some embodiments, each baseline serving may include ingredients in the following quantities:

1. a protein source in the amount of 14 g to 128 g;

2. a fat source in the amount of 4 g to 42 g;

3. a carbohydrate source in the amount of 25 g to 80 g;

4. a vegetable source in the amount of 40 g to 128 g.

In some embodiments, each baseline serving may include ingredients in the following quantities:

1. a protein source in the amount of 28 g to 256 g;

2. a fat source in the amount of 4 g to 42 g;

3. a carbohydrate source in the amount of 50 g to 160 g;

4. a vegetable source in the amount of 40 g to 128 g.

In some embodiments, each baseline serving may include ingredients in the following quantities:

1. a protein source in the amount of 28 g to 256 g;

2. a fat source in the amount of 4 g to 42 g;

3. a carbohydrate source in the amount of 50 g to 160 g;

4. optionally, a vegetable source in the amount of 40 g to 128 g.

In some embodiments, each baseline serving may include ingredients in the following quantities:

1. a protein source in the amount of 28 g to 256 g;

2. a fat source in the amount of 4 g to 42 g;

3. a carbohydrate source in the amount of 25 g to 80 g;

4. a vegetable source in the amount of 80 g to 256 g.

In some embodiments, each baseline serving may include ingredients in the following quantities:

1. a protein source in the amount of 14 g to 128 g;

2. a fat source in the amount of 4 g to 42 g;

3. a carbohydrate source in the amount of 25 g to 80 g;

4. a vegetable source in the amount of 80 g to 256 g.

In some embodiments, each baseline serving may include ingredients in the following quantities:

1. a protein source in the amount of 28 g to 256 g;

2. a fat source in the amount of 8 g to 84 g;

3. a carbohydrate source in the amount of 50 g to 160 g;

4. a vegetable source in the amount of 80 g to 256 g.

The protein may be obtained from food sources known to be high in protein. Specifically, the protein source may be selected from lean meat, such as chicken without skin, beef, pork and fish. Alternatively, the protein source may be selected from a variety of beans to create a vegetarian friendly bulk food product. In some embodiments, the protein source may include, but not limited to, beef, bison, buffalo, chicken, duck, goose, hen, lamb, pork, quail, turkey, veal, venison, crab, bass, catfish, clams, cod, flounder, grouper, halibut, haddock, lobster, mussels, oysters, rockfish, salmon, scallops, shrimp, snapper, swordfish, trout, tuna, cheese (all natural), cottage cheese, cream cheese, eggs, milk (whole), yogurt (plain), raw nuts (all varieties), seeds (pumpkin sunflower), seeds (hemp, chia, etc.), tempeh, or tofu.

In some embodiments, the protein may have a serving size. In some embodiments, the protein serving size is in a range from about 14 g to about 128 g. In some embodiments, the protein serving size is in a range from about 28 g to about 256 g. In some embodiments, the protein serving size is in a range from about 50 g to about 85 g. In some embodiments, the protein serving size is in a range from about 32 g to about 128 g. In some embodiments, the protein serving size is in a range from about 14 g to about 42 g. In some embodiments, the protein serving size is 50 g. In some embodiments, the protein serving size is 85 g. In some embodiments, the protein serving size is in a range from about 1 ounce to about 3 ounces. In some embodiments, the protein serving size is in a range from about 2 ounces to about 6 ounces. In some embodiments, the protein serving size is in a range from about 2 ounces to about 3 ounces. In some embodiments, the protein serving size is in a range from about 1 ounce to about 2 ounces. In some embodiments, the protein serving size is 3 ounces. In some embodiments, the protein serving size is 2 ounces. In some embodiments, the protein serving size is in a range from about ¼ cup to about 1 cup. In some embodiments, the protein serving size is in a range from about ½ cups to about 2 cups. In some embodiments, the protein serving size is in a range from about ½ cup to about 1 cup. In some embodiments, the protein serving size is about ½ cup. In some embodiments, the protein serving size is in a range from about 1 tablespoon to about 3 tablespoons. In some embodiments, the protein serving size is in a range from about 2 tablespoons to about 6 tablespoons. In some embodiments, the protein serving size is in a range from about 2 tablespoons to about 3 tablespoons. In some embodiments, the protein serving size is 2 tablespoons. In some embodiments, the protein serving size is 3 tablespoons. In some embodiments, the protein serving size is 1 egg.

The vegetable source may be obtained from any vegetable source including, but not limited to, carrot, spinach and other vegetables. In some embodiments, the vegetable source may include, but not limited to, alfalfa sprouts, artichokes, arugula, asparagus, bean sprouts, beets, bok choy, broccoli (all varieties), Brussels sprouts, cabbage (green), cabbage (red), carrots, cauliflower, celery, collard greens, cucumber, eggplant, fennel, green beans, kale, leeks, lettuce, mushrooms, okra, onion, parsnips, peppers (green), peppers (all other), pumpkin, radish, sauerkraut, spinach, squash (all varieties), Swiss chard, snow peas, turnips, zucchini, avocado, tomatoes, tomato sauce, olives, corn, or peas.

In some embodiments, the vegetable may have a serving size. In some embodiments, the vegetable serving size is in a range from about 40 g to about 128 g. In some embodiments, the vegetable serving size is in a range from about 80 g to about 256 g. In some embodiments, the vegetable serving size is in a range from about 64 g to about 128 g. In some embodiments, the vegetable serving size is about 64 g. In some embodiments, the vegetable serving size is about 128 g. In some embodiments, the vegetable serving size is in a range from about ½ cup to about 1 cup. In some embodiments, the vegetable serving size is in a range from about 1 cup to about 2 cups. In some embodiments, the vegetable serving size is about ½ cup. In some embodiments, the vegetable serving size is about 1 cup. In some embodiments, the vegetable serving size is 8 olives. In some embodiments, the vegetable serving size is 16 olives.

The vegetable source may comprise multiple vegetables including, but not limited to, carrots, spinach and other green vegetables; the starch group includes rice, potato and yam (sweet potato), or a combination thereof; and the protein group includes chicken, beef, lamb, pork, fish and beans (vegetarian) or a combination thereof. These foods are by way of example only and are non-limiting. The ingredients can be selected to create a variety of different food products, having the same nutritional composition, e.g. a sweet food product can be created by combining ingredients which include coconut, beans, quinoa, hemp, raw cocoa, squash or other sweet vegetables.

The carbohydrate may be obtained from any starch source including, but not limited to, potatoes, rice and yams (sweet potato). In some embodiments, the carbohydrate source may include, but not limited to, apple, apricot, banana, blackberries, blueberries, cantaloupe, cherries, grapefruit, grapes, guava, honeydew, kiwi, mango, nectarine, orange, papaya, peach, pear, pineapple, plantain, plum, raspberries, strawberries, watermelon, dried fruit, potatoes, sweet potatoes, yams, amaranth, barley, bread, brown rice, corn tortilla, wheat tortilla, couscous, farro, millet, spelt, oatmeal, pasta, quinoa, jicama, whole wheat, coconut flour, beans (all varieties), edamame, hummus, lentils, or lima beans.

In some embodiments, the carbohydrate may have a serving size. In some embodiments, the carbohydrate serving size is in a range from about 25 g to about 80 g. In some embodiments, the carbohydrate serving size is in a range from about 50 g to about 160 g. In some embodiments, the carbohydrate serving size is in a range from about 32 g to about 64 g. In some embodiments, the carbohydrate serving size is 32 g. In some embodiments, the carbohydrate serving size is 64 g. In some embodiments, the carbohydrate serving size is in a range from about ¼ cup to about ½ cup. In some embodiments, the carbohydrate serving size is in a range from about ½ cup to about 1 cup. In some embodiments, the carbohydrate serving size is about ¼ cup. In some embodiments, the carbohydrate serving size is about ½ cup. In some embodiments, the carbohydrate serving size is 1 corn or wheat tortilla. In some embodiments, the carbohydrate serving size is 1 slice of bread.

The fat from the fat source may be obtained from any fat source including, but not limited to, butter, coconut oil, oils (olive, walnut, sesame, or avocado), homemade dressing, or unsweetened coconut flakes.

In some embodiments, the fat may have a serving size. In some embodiments, the fat serving size is in a range from about 4 g to about 42 g. In some embodiments, the fat serving size is in a range from about 14 g to about 42 g. In some embodiments, the fat serving size is in a range from about 1 teaspoon to about 2 teaspoons. In some embodiments, the fat serving size is about 1 teaspoon. In some embodiments, the fat serving size is about 2 teaspoons. In some embodiments, the fat serving size is in a range from about 1 tablespoon to about 3 tablespoons. In some embodiments, the fat serving size is in a range from about 1 tablespoon to about 2 tablespoons. In some embodiments, the fat serving size is in a range from about 2 tablespoons to about 3 tablespoons. In some embodiments, the fat serving size is about 1 tablespoon. In some embodiments, the fat serving size is about 2 tablespoons. In some embodiments, the fat serving size is about 3 tablespoons.

Table 2 provides further information on the composition of the food product. The portion size of a single serving of food product, according to Table 2 is equal to 450 grams which is approximately 16 ounces or 2 cups of food which would be regarded as a good size meal to satisfy most people. The caloric value of 450 calories is sufficient to sustain a person between meals, depending on activity.

Table 2 shows that each ingredient, selected as being representative of one of the essential nutrients, includes trace amounts of the other essential nutrients. For example chicken, which has a high protein content and is therefore selected to represent this nutrient in the meal portion, has some fat content. Similarly a vegetable, such as spinach, includes some protein, fat and fiber in addition to its carbohydrate content. The essential nutrient content for each selected ingredient is taken into consideration when preparing the food product to ensure that the defined proportions are closely adhered to.

Carbohydrates, consumed alone, cause insulin spikes which lead to cravings and hunger pangs. For this reason, it is important to consume carbohydrates in combination with protein and fiber, to control the insulin spikes.

The nutritional compositions of most foods are known or determinable and a comprehensive chart showing nutritional breakdown of all foods is therefore not included in this specification.

Table 3 shows the recommended percentage content for protein, carbohydrate, fiber and fat respectively, based on 2000 calories of the food product of the invention.

From Tables 2 and 3 it can be seen that a consumer eating 2000 calories of the food product will consume fiber and essential nutrients in quantities that vary from the RDA. Specifically Tables 2 and 3, when compared to Table 1, highlight the dominance, recommended by the USDA, of carbohydrates and fat (83%) with fiber and protein representing only 17%. The food product of the invention requires that the fiber and protein contents should be more than the USDA recommendations, and that the fat and carbohydrate contents should be less than the USDA recommendations. It is believed that these ratios are more conducive to managing insulin levels in a healthy manner.

A chosen quantity of the mixture, prepared in accordance with the aforementioned requirements, sufficient, say, for 8 to 24 meals, is packed into a container. A scoop or cup, of a defined size, can be provided so that a consumer can make an accurate measurement of a food portion or a meal—by way of example a standard measuring cup would contain one half of the recommended size of a meal portion of the food product.

After packaging, the container and its contents are snap frozen or refrigerated.

The bulk food product can be distributed (sold) in any convenient manner. The product lends itself to sales via online orders, using appropriate software which is linked to the software referred to in the earlier specification, and can be delivered to a consumer's residence, work place or other specified address. The food product could also be delivered to retail outlets in bulk for resale by retailers or the recipe and certain portions of the food product could be provided to retailers or restaurants etc. for preparation and sale under license.

The food product that is frozen or refrigerated can be packaged and shipped with sufficient ice packs to allow for the food product to arrive in 1 to 3 days frozen or refrigerated.

Meals, in one or more portions for one or more people, can be consumed cold or can be microwaved or conventionally heated prior to consumption.

The invention thus extends to a bulk food product which is prepared from a mixture of primary ingredients which are selected using the food index system referred to in the earlier specification, in sufficient quantities to make up at least one meal, but preferably a plurality of meals. The mixture includes portions, or parts of portions, of fiber and essential nutrients (i.e. protein, carbohydrate and fat) and additional proprietary ingredients.

One significant benefit lies in the fact that the bulk food product offers a commercially effective and convenient manner of adapting and implementing a weight management regime in terms of which a consumer establishes his/her daily base food allowance, in a defined unit system, and selects food, which is to consumed, from an established food value index, listing food values specified in the same unit system. It is recommended that a person should consume from 90% to 110% of his daily base food allowance for a healthy and effective weight management plan.

In the food value index system shown in Table 4 food varieties are divided into four categories comprising vegetables, protein, carbohydrate and fat. Each category is divided into sub-categories as follows: vegetables are divided into green vegetables and other vegetables (red, white, orange); protein sources are divided into egg, fish/shellfish, white meat, red meat, dairy and raw nuts; carbohydrate sources are divided into berries, other fruit, starch and grains; fat sources are divided into oils, salad dressing and butter. Each sub-category is assigned a value which is referred to as the “food value”, represented in units.

Table 4 reflects portion sizes (third column) that are allocated to each sub-category of food, and food values (fourth column) associated with each portion. The food values of Table 4 can be used in the method of the invention according to the following example: a portion (1 cup) of green vegetables has a food value of 10 units; two portion (6 ounces) of white meat has a food value of 80 units; one portion of fat (1 teaspoon) has a food value of 20 units; and one portion (½ cup) of starch has a food value of 40 units. Each food value for the corresponding portion is based on the following: calorie content (energy value), sugar content (glucose and insulin effect), fiber content, protein content, fat content and processed content (additives, refined sugars, and health benefit, whether positive or negative).

A general formula for determining the food value (FV) for each type of food is as follows.

For a given amount of the food, i.e. a food portion expressed in a conventional measurement unit (a cup, an ounce, a tablespoon, millilitres, grams or the like), the following assessments are made:

1. a base energy value (B) is determined by dividing the calorie value of the food portion by 3;

2. the following values, for the chosen food portion, are established in grams;

2.1 fiber content

2.2 protein content (P);

2.3 sugar content (S);

2.4 fat content (FT); and

2.5 processed content (PC).

The food value for the chosen food portion is established by subtracting the positive values items (fiber and protein) from the base energy value (B) and by adding the negative value items (sugar, fat and processed content) to the base energy value (B), as follows:

FV=B−F−P+S+FT−PC

The values may be rounded for simplicity.

The formula has been established and proven, by trial and experimentation, as an effective basis for implementing the method of the invention.

Clearly it is possible to scale the food value (FV) up or down, to a convenient-to-use figure, by correspondingly increasing or decreasing the size of a portion.

When using the formula, the food value, FV (in units), per food type is generally equated to between 2.7 and 3 calories per unit. As an example: the food value of the meal described above, of 150 units, equates to about 450 calories at a ratio of 3 calories per food value unit. If used for 3 meals, as a daily example, this equates to 450 units and approximately 1,350 calories.

Table 5 contains a listing which allows a consumer to perform a metabolic calculation to determine his or her daily base food allowance in terms of units which correspond to the units used to represent the food values in Table 4.

The base food allowance is linked to the following parameters, namely: the consumer's gender, age, body mass index (BMI) and life activity, as set out in a first column of Table 5 Other parameters, such as the consumer's height, weight and waist circumference can be used as an alternative to the consumer's BMI. A second column of Table 5 contains, in respect of each of the aforementioned parameters, the number of units in the corresponding base food allowance.

A third column in Table 5 (headed BFA calculation) allows a consumer to enter the number of units which apply to him/her. For example, a male consumer in the age group of 51 to 60, with a BMI score of 36+, and with a medium life activity index, has a base food allowance of 470 units.

The values in Table 5 are used to establish whether a consumer should optimally consume more or fewer food value units per meal, or per day. A formula is used to generate a personalized base food allowance value (BFA) which is regarded as the ideal food consumption for a consumer using the food value index before any exercise activity.

It is generally established that males have higher base metabolic rates than females and, as such, gender is a dominant variable in determining the BFA. The value assigned to a consumer based on gender is known as the base allowance.

A consumer's metabolism slows down with age. In Table 5, the scale typically has a 5 point drop by decade. Exceptions are for the decades 31 to 40, and 41 to 50. In these age brackets there is a 10 point drop to accommodate the known drop in metabolism from age 35.

The higher a consumer's body mass index (BMI) the less his metabolism performs and (generally) the more weight the consumer needs to lose. BMI is calculated by dividing the consumer's weight in kgs, by his height in meters squared. By way of example, a person who is 1.8 m tall and who weighs 80 kg has a BMI of about 24.7=(80/[1.8]2). The BMI calculation could be replaced with other body mass composition estimates such as waist circumference.

Life activity relates to a consumer's energy requirements based on lifestyle and work activity before exercise activity. As an example, a 65 year old male with a BMI of 36 and with a sedentary lifestyle has a BFA (base food allowance) of 455. A 20 year old male with a BMI under 20 and with high life activity has a BFA of 530.

To avoid exercise-induced muscle loss or muscle loss due to an insufficient energy intake, an exercise activity index is employed to increase the food allowance of a consumer, based on exercise activity. The increased food allowance also serves to motivate the consumer to be active-see Table 6.

Table 6 shows the activity index, in units which correspond to the units which apply to Table 4 and Table 5. The activity index is determined in accordance with exercise type i.e. strength training, high intensity interval training/endurance training and cardio (constant-medium intensity), and the duration of the exercise. Each exercise type is further subdivided into more specific categories of exercise.

Exercise value points are based on the duration of performing a particular exercise. The duration is measured in 5 minute time intervals with each subsequent 5 minute interval having a higher value than the previous 5 minute interval. The incremental increase in exercise value encourages exercise for longer intervals.

If a consumer performs a particular exercise for a defined time interval, the value determined by the activity index can be added to the BFA, increasing the daily food allowance of the consumer.

Thus, with reference to Table 6, if a consumer does strength training for 20 minutes together with 15 minutes of steady walking, the activity index for that consumer per day is 85 units.

The use of the bulk food product can be controlled directly, e.g. by a consumer who follows a chart, tables or the like.

The weight management method can, however, be implemented using software which enables a consumer to create a profile which includes all relevant personal information, and his particular weight management goal (i.e. weight loss, weight gain or weight maintenance). The software uses this information to calculate the consumer's daily base food allowance and to log daily food consumption and activities.

In a preferred implementation of the method use is made of a computer link, e.g. the internet, which allows the consumer to interact individually with a program located at a suitable server.

The consumer, once authorised, is guided to register an online profile. The consumer is prompted to enter information in accordance with predetermined parameters and in response thereto a calculation is done to determine the consumer's base food allowance.

The consumer is subsequently prompted to enter information on the consumer's activities on a daily or other regular basis. Each activity which is logged is influenced by the activity index.

Alternatively or additionally the program, in response to data input by the consumer, generates an activity program suited for the consumer which, inherently, will lead to the allocation of units, associated with the activity index, to the consumer's daily food allowance.

In response to the determined base food allowance and logged activities the program then outputs the daily food allowance for the consumer which is the sum of the daily base food allowance (in units) and allocated units due to activities performed, where the units are provided by the activity index. This information is used to determine at least the size of each portion of the bulk food product which is thereafter supplied to the consumer.

In any weight management program motivation is a key ingredient to success. Motivational factors are introduced by the program and presented to the consumer in various forms, e.g. a message which brings home to the consumer the importance of managing food intake using the bulk food product of the invention.

Additionally, a results-tracking program is started and updated regularly. This allows the consumer to have a personal assessment, commencing at a starting point, which includes data such as the consumer's starting weight, starting waist measurement, base food allowance and activity index. The daily food allowance may be updated as consumed food and/or activities are logged and notification of the updated daily food allowance is given to the consumer by the program.

Other information of interest, primarily related to motivational aspects, may be included and displayed at appropriate intervals to the consumer. For example data on the consumer's goal weight, metabolic age estimate, stress and emotional eating score, and processed food score may be included.

A primary reason behind the use of the personal assessment program lies in the fact that it provides a ready means for implementation of the weight management method of the invention. This, however, is done in a way which motivates the consumer to achieve targets and to become aware of the effect that the intakes of different types of food could have on the consumer's body. Ultimately the use of the personal assessment program helps the weight management method to be implemented in a reasonable and not in a stressful manner and leads the consumer to a healthy eating style which can readily be maintained.

A primary objective of the food product is to enable the consumer to eat balanced meals of healthy foods without suffering from hunger pangs, food cravings, and isolation from family and friends, particularly at meal times. In the last-mentioned respect it is recognised that social considerations can weigh heavily on the effectiveness of a weight management regime and, taken in isolation, a weight management program which a consumer regards as antisocial could cause the consumer to abandon the program in its entirety.

Essentially the invention enables the consumer to balance energy in against energy expended with a difference which is linked to weight loss, weight gain, or weight maintenance, as the case may be. This allows the consumer to achieve weight management on a consistent and sustainable basis. The food groupings and portion control allow the consumer to maintain a high energy level. Risks which are associated with diabetes and heart disease and which are linked to the intake of certain food types are reduced in the management method of the invention.

The provision of the bulk food product substantially simplifies the weight management process, be it for weight gain, weight loss or weight maintenance purposes. The bulk food product, prepared under tightly controlled conditions provides one or more meals, in bulk form, with each meal having a fully balanced composition derived from fibre and essential nutrients i.e. protein, carbohydrate and fat.

For inherently different flavors the bulk food product can be provided with a chicken base, a beef base, a vegetarian base, or the like. Supplements consisting for example of stir fried vegetables can be added to a meal portion.

The utility and efficacy of the composition of the bulk food product have been demonstrated in a trial in which 33 subjects participated in a weight loss program. The effects of the program on each subject were tracked for a period of 29 weeks. Subjects were required to create meals based on the food product and to consume the food product in quantities corresponding to their daily food allowances as described above. Subjects also were required to participate in an exercise regimen.

On average the 33 subjects were 74% compliant in respect of food intake and 75% compliant in respect of exercise, i.e. activity index. In particular the subjects consumed, in food terms, approximately 600 units per day (this is about 1700 calories per day) before exercise and before taking into account the “bonus” units associated with each person's activity index. Each person on average exercised for 90 minutes per week.

Despite a calorie intake which was meaningfully larger than the intake allowed by a calorie-restrictive diet, the subjects reported an average weight loss of 32 lbs over the 29 week test period, i.e. a weight loss of slightly more than 1 lb per week. The lowest weight loss over the 29 week period of a subject was 11 lbs and the highest weigh loss of a subject was 49 lbs.

There was a significant reduction (6 inches) in average waist measurement and the average reduction in body mass index was 15%.

The subjects ate wholefoods, real fats and sugar and minimised the intake of low fat and sugar substitutes. The subjects stabilised their insulin levels, reducing their triglyceride indexes by 28% and blood glucose levels by 9% with all risk factor indicators lowering significantly.

20 of the 33 participants had metabolic syndrome, i.e. they were at risk of diabetes and heart disease, at the start of the trial. At the end of the 29 week period only 2 of these participants had metabolic syndrome and, as a group, risk factors in total were reduced by 64%.

75% of the subjects who were on cholesterol-lowering medications were able to eliminate such medications. 67% eliminated their diabetes medications and 50% eliminated blood pressure medications.

The bulk food product of the invention frees a consumer from selecting ingredients and making up balanced meals. Minimal effort and work are required by a consumer, using the bulk food product of the invention, to implement a weight loss, weight gain or weight maintenance program, in a cost effective, safe and healthy manner without harmful side effects.

The balanced nature, high protein and fiber levels and recommended portion sizes help to manage insulin levels/insulin resistance, and sustain a consumer for from 3 to 5 hours. The food product can also be used effectively in shelters or could be shipped to third world countries to treat malnutrition in a cost effective manner.

TABLE 2
Food Product Nutritional Panel

| | | | | | Nutritional Value | | | | |
|---|---|---|---|---|---|---|---|---|---|
| | | | | Food | Protein | Fat | Carb | Fiber | |
| | Ounces | Grams | Calories | Value | Grams | Grams | Grams | Grams | Total |
| Food Product | | | | | | | | | |
| Lean Chicken ( white) | 2.75 | 77.96 | 128.3 | 40 | 24.18 | 2.79 | 0.00 | 0.00 | |
| Bean | 2.00 | 56.70 | 72.5 | 32 | 4.26 | 0.06 | 14.04 | 4.14 | |
| TOTAL PROTEIN | 4.75 | 134.66 | 200.8 | 72 | 28.44 | 2.85 | 14.04 | 4.14 | |
| Carrots | 2.00 | 55.70 | 20.0 | 5 | 0.00 | 0.00 | 4.00 | 2.00 | |
| Spinach | 3.00 | 85.05 | 20.0 | 4 | 2.53 | 0.22 | 3.19 | 2.03 | |
| Squash | 1.50 | 42.53 | 12.0 | 4 | 0.28 | 0.11 | 2.75 | 0.60 | |
| Sub Total Carbohydrates (vegetables) | 6.50 | 184.28 | 52.0 | 13 | 2.81 | 0.33 | 9.94 | 4.63 | |
| Carbohydrates (starch such as potato) | 4.00 | 113.40 | 64.0 | 40 | 1.16 | 0.12 | 15.00 | 2.13 | |
| TOTAL CARBOHYDRATES | 10.50 | 297.68 | 116.0 | 53 | 3.97 | 0.45 | 24.94 | 6.76 | |
| FAT (oil) | 0.40 | 11.34 | 95.0 | 15 | 0.00 | 10.80 | 0.00 | 0.00 | |
| PHYTO BLEND | 0.29 | 8.22 | 30.0 | 8 | 3.20 | 1.00 | 4.35 | 3.35 | |
| PRESERVATIVE-FLAVOR BLEND | 0.04 | 1.05 | 8.0 | 2 | 0.30 | 0.10 | 1.40 | 0.35 | |
| One Serving of the Food Product | | | | | | | | | |
| 1 Serving of Food Product | 16.0 | 452.9 | 450 | 150 | 35.91 | 15.20 | 44.73 | 14.60 | 110.44 |
| | | | | | 32.5% | 13.8% | 40.5% | 13.2% | 100.0% |
| Nutritional Grams Per Calorie | | | | | 0.0798 | 0.0338 | 0.0995 | 0.0325 | |
| Food Product | | | | | | | | | |
| Daily RDA Nutritional Percentage | | | | | 71.8% | 23.4% | 14.9% | 58.4% | |
| Food Product Based On 2000 Calorie Diet | | | | | | | | | |
| Food Product Values for 2000 Calories | | | 2.000 | | 159.7 | 67.6 | 198.9 | 64.9 | 491.10 |
| Content Percentage | | | | | 32.52% | 13.76% | 40.50% | 13.22% | 100.0% |
| Vs. Recommended RDA | | | 2.000 | | 50.00 | 65.00 | 300.00 | 25.00 | 440.00 |
| | | | | | 11.36% | 14.77% | 68.18% | 5.68% | 100.0% |
| Difference Food Product vs. RDA | | | 2.000 | | 109.7 | 2.6 | −101.1 | 39.9 | 51.10 |
| | | | | | 68.7% | 3.8% | −50.8% | 61.5% | 10.4% |
| Phyto Blend | | | | | | | | | |
| Flax | 0.05 | | 5.00 | | 0.20 | 0.50 | 0.35 | 0.35 | |
| Wheat Grass | 0.14 | | 15.00 | | 1.00 | 0.00 | 2.00 | 2.00 | |
| Other Phyto Foods | 0.05 | | 5.00 | | 1.00 | 0.50 | 2.00 | 1.00 | |
| Enzymes and Probiotics | 0.05 | | 5.00 | | 1.00 | 0.00 | 0.00 | 0.00 | |
| Phyto Total | 0.29 | | 30.00 | | 3.20 | 1.00 | 4.35 | 3.35 | |
| Preservative-Flavor Blend | | | | | | | | | |
| Sea Salt: sodium | 0.01 | | 0.00 | | 0.00 | 0.00 | 0.00 | 0.00 | |
| Lemon | 0.01 | | 0.35 | | 0.00 | 0.00 | 0.10 | 0.00 | |
| Rosemary | 0.01 | | 1.85 | | 0.05 | 0.10 | 0.30 | 0.20 | |
| Garlic | 0.01 | | 4.65 | | 0.25 | 0.00 | 1.00 | 0.15 | |
| Apple Cider Vinegar | 0.01 | | 0.00 | | 0.00 | 0.00 | 0.00 | 0.00 | |
| Preservative-Flavor Total | 0.05 | | 6.85 | | 0.30 | 0.10 | 1.40 | 0.35 | |

TABLE 3

| Food Group | Weight in grams | Percentage content |
|---|---|---|
| Carbohydrates | 199 | 40% |
| Protein | 160 | 33% |
| Fiber | 65 | 13% |
| Fat | 68 | 14% |
| Total | 492 | 100% |

TABLE 4

| | Examples or Recommendations | Portion | Food Values |
|---|---|---|---|
| Vegetables | | | |
| Green (Cooked or ram) | Broccoli, spinach, green beans | 1 cup | 10 |
| Other (Red, white, orange) | Cauliflower, Carrots | 1 cup | 20 |
| Protein | | | |
| Egg | | 1 | 25 |
| Fish/Shellfish | Salmon, scallops | 3 oz | 35 |
| White meat | Chicken, turkey, pork | 3 oz | 40 |
| Red meat | Beef, lamb | 3 oz | 45 |
| Dairy | Milk, yogurt, cheese | 4 oz or ½ cup | 45 |
| Raw Nuts | Walnus, Almonds | 3 oz or 1/3 cup | 50 |
| Carbohydrates | | | |
| All Berries | Blueberries, cherries | ½ cup | 40 |
| All other fruit | Apple, banana | 1 med or ½ cup | 50 |
| Starch | Potato, sweet potato | 1 med or ½ cup | 40 |
| Grains | Brown rice, quinoa, multi-grain bread | ½ cup or 1 slice | 45 |
| Fat | | | |
| Oils (Olive, Coconut) | | 1 tbsp. | 10 |
| GOLO salad dressing | | 2 tbsp. | 15 |
| Butter | | 1 teaspoon | 20 |
| Limit | | | |
| Natural condiments | Soy sauce, mustard | 1 tbsp | 25 |
| Sugars | Honey, syrup, sugar, jam | 1 teaspoon | 25 |
| Dark chocolate | | 2 oz | 40 |
| Peanut butter | | 1 tbsp. | 50 |
| Beer | | 8 oz | 75 |
| Wine | | 4 oz | 75 |
| Hard liquor | | 2 oz | 75 |
| Bacon/Sausage | | 2 oz, 1 link | 75 |
| Deli meat | | 2 oz | 75 |
| Avoid | | | |
| Processed Condiments | Bottled dressings, marinades, ketchup, etc. | 1 tbsp | 35 |
| Artificial sweeteners | | 1 tsp/1 packet | 50 |
| Processed Beverages (diet) | Soda, juice, coffee drinks, etc. | 8 oz | 75 |
| Processed snacks or desserts | Cookies, chips, ice cream, crackers, etc. | 1 serving | 100 |
| White flour products | Breads, pasta | 1 serving | 100 |
| Processed Beverages (reg) | Soda, juice, coffee drinks, etc. | 8 oz | 150 |
| All processed, packaged food | Frozen meals, boxed rice, cereal, etc. | 1 serving | 150 |
| Protein bars/shakes | | 1 shake/1 bar | 150 |
| Mixed drinks w/alcohol | | 1 drink | 150 |
| All Fast. convenience foods | Burgers, fries, pizza, fried chicken, etc. | meal | 175 |

TABLE 5

| | | BFA |
|---|---|---|
| | | CALCULATIONS |
| Gender | | |
| Male | 390 [symbol] | 396 |
| Female | 368 | |
| Age | | |
| <20 | 50 | |
| 21-30 | 45 | |
| 31-40 | 40 | |
| 41-50 | 30 | |
| 51-60 | 25 [symbol] | 25 |
| 61+ | 20 | |
| Body Mass | | |
| Composition Estimate | | |
| <20 | 40 | |
| 21-28 | 35 | |
| 29-35 | 25 | |
| 36+ | 20 [symbol] | 20 |
| Life Activity | | |
| (not excercise) | | |
| Low (sedentary) | 20 | |
| Medium (some gardening- | 35 [symbol] | 35 |
| walking, ect.) | 50 | |
| High (physical job) | | |
| Base Food Allowance | | 476 |

[symbol]

indicates data missing or illegible when filed

TABLE 6

| | 5 | 10 | 15 | 20 | 25 | 30 |
|---|---|---|---|---|---|---|
| | Minutes | Minutes | Minutes | Minutes | Minutes | Minutes |
| Strength Training | | | | | | |
| Spot Training | 13 | 26 | 41 | 55 | 70 | 86 |
| Strength Training (maching-free | 15 | 30 | 46 | 63 | 80 | 97 |
| weights-TRX etc.) | | | | | | |
| Circuit Training (strength | 14 | 29 | 44 | 60 | 77 | 94 |
| training with cardio intervals) | | | | | | |
| High Intensity | | | | | | |
| Interval/Endurance Training | | | | | | |
| Burst High Intensity Training | 12 | 26 | 39 | 54 | 68 | 83 |
| Stair Climbing | 11 | 22 | 34 | 47 | 59 | 72 |
| Endurance (rowing, swimming) | 10 | 22 | 34 | 47 | 59 | 72 |
| High Intensity Workout Classes | 11 | 23 | 35 | 48 | 61 | 75 |
| (Zumba, etc.) | | | | | | |
| High Intensity Walking | 11 | 23 | 35 | 48 | 61 | 75 |
| High Intensity Sports | 10 | 20 | 31 | 42 | 54 | 66 |
| (soccer/tennis, etc.) | | | | | | |
| Cardio (constant-medium | | | | | | |
| intensity) | | | | | | |
| Cardio Machine | 7 | 15 | 22 | 30 | 39 | 47 |
| Steady Walking | 7 | 15 | 22 | 30 | 39 | 47 |
| Yoga | 7 | 15 | 22 | 30 | 39 | 47 |
| Low Intensity Sports (golf) | 7 | 15 | 22 | 30 | 30 | 47 |

This invention and embodiments illustrating the method and materials used may be further understood by reference to the following non-limiting examples.

### Example 1

### Effect of the GOLO Diet (Weight Management Plan) and GOLO Release Supplement (Weight Loss Composition) on Weight, Glycemic Control and Indicators of Insulin Sensitivity in Overweight and Obese Patients with Type 2 Diabetes

Summary: 26 subjects with type 2 diabetes mellitus were recruited in an open-label study of the GOLO Diet with Release supplement at a single clinical site. 15 subjects completed the study over the 13-week treatment period. Overall weight loss averaged 7.9 lbs (−3.8%) and BMI levels dropped by 1.3 (−3.7%). Markers of glycemic control improved with Hemoglobin A1C (−9.2%) and fasting blood glucose (−17.9%) decreasing while markers of insulin resistance including insulin levels (−18.7%) and HOMA-IR (−36.6%) showed substantial improvement. Favorable improvement was also noted in other laboratory results and clinical measures including cholesterol levels and inflammation.

Introduction: Excess body weight from overeating, poor nutrition and lack of exercise is highly correlated with health status. Clinical weight loss in overweight and obese people is associated with improvements in clinical markers of health, including key measures of blood sugar and blood lipids used to determine a person's health status.

Populations who are overweight and obese include people who exhibit a wide range of blood sugar levels, ranging from healthy to pre-diabetic to type 2 diabetes. Overweight and obese people on this spectrum often have difficulty obtaining meaningful or sustained weight loss. Body weight is also a leading indicator of high blood sugar levels. It is well established that people who are healthy, pre-diabetic or diabetic who lose weight and exercise tend to lower their measures of blood sugar and blood lipids, and are more likely to improve their health status (1).

GOLO has created a weight management program that includes a supplement (weight loss composition) known as Release and (GWMP) and is designed to help people who are overweight or obese get the proper balance of macro and micro nutrients from conventional foods, in the proper portions and combinations to help keep insulin steady for weight loss, and help them to transition to a healthier lifestyle. The GWMP includes a point-based system from the four macronutrient food groups that is based on the individual's activity level and body mass. Additionally, the program includes common-sense instructions, motivation and tips supporting compliance and recommends a minimum of 15 minutes of exercise per day. The Release dietary supplement contains 7 plant based ingredients and 3 minerals including zinc and chromium, essential nutrients that support regulation of blood sugar.

In case studies from clinician and wellness program use, GWMP has shown the ability to reduce body weight in both healthy and diabetic people who are overweight or obese. Secondary endpoints including measures of blood sugar have been observed to decrease as a result of the GWMP program (2).

While the GWMP has been developed and used in private clinical practice in both healthy overweight and type 2 diabetic people, more systematic research is needed to determine to what extent it is able to support healthy weight loss. This open-label pilot study is intended to observe the effects of the program in a representative group of subjects with stable type 2 diabetes at one outpatient medical practice.

Study design: This observational study evaluating the effect of the GOLO Weight Management Program (GWMP) on weight and metabolic syndrome indicators in overweight or obese subjects with stable type 2 diabetes mellitus was conducted at one clinical site in the United States. The study consisted of 4 visits over approximately 13 weeks.

At visit 1, study eligibility was determined and subjects were given the commercially available GOLO Weight Management Program and instructed on the program's diet and exercise guidelines. Subjects were given the Release Supplement and instructed to take one capsule three times a day with meals. Laboratory and body measurements were obtained. Visit 2 was a telephone call to subjects to assess tolerability of the program. At approximately week 4, subjects returned for Visit 3 for a compliance and tolerability assessment and body measurements. The final visit 4 occurred at approximately week 13 and included body measurements, laboratory analysis and compliance and tolerability assessment.

Body measurements included fasting weight, height, waist and hip circumference, and resting blood pressure and pulse were taken at each visit. A Tanita scale was used to calculate BMI, body fat and visceral fat. Laboratory evaluation included hemoglobin A1C, fasting insulin, fasting blood glucose, lipid panel, metabolic panel including liver testing, hsCRP, CBC, sex hormones, and PSA in men. Homeostatic model assessment of insulin resistance HOMA-IR was calculated using the formula (Fasting Blood Glucose) (Fasting Insulin)/22.5(3). Stress and Anxiety were measured at visit 1 and visit 4 using a standardized Stress Questionnaire.

Subjects: 26 subjects consented to participate in the study. One subject screen failed for a BMI outside of inclusion criteria. 10 subjects withdrew or were removed from the study for the following reasons: 5 lost to follow-up/voluntarily withdrew, 3 adverse events and 2 poor study compliance. 15 subjects completed the study and attended all 4 visits.

Results: 5 Males and 10 Females completed the study. The average age of males was 57.8 and females 58.5. The average starting weight was 206.3 pounds and BMI 34.3 which is considered obese. Average hemoglobin A1c (7.5) and fasting blood glucose (153.7) were elevated at baseline indicating poorly controlled type 2 DM. Initial resting blood pressure (124.1/75.5) and LDL cholesterol (100.5) were already at or near goal levels in this group of diabetic subjects, primarily due to pre-study treatment with blood pressure and cholesterol medications.

Changes from baseline visit 1 to visit 4 in weight, BMI, body analysis and body measurements are listed in Table 7. Overall weight loss averaged 7.9 lbs (−3.8%) and BMI levels dropped by 1.3 (−3.7%). Loss of Fat Mass (−7.2%) were more pronounced in this overweight/obese population. Waist measurements (−6.1%) improved more than hip measurements (−3.5%) resulting in a favorable change in waist/hip ratio (−6.1%). Changes in resting blood pressure and pulse were minimal as the majority of subjects were treated with blood pressure medication as is standard care in diabetic patients. Changes in the Stress/Anxiety survey were substantial (−49.0%).

TABLE 7
CHANGES IN WEIGHT AND BODY MEASUREMENTS

| | Visit 1 | Visit 4 | |
|---|---|---|---|
| | (Week 1) | (Week 13) | Change |
| Weight (lbs.) | 206.3 | 198.4 | −7.9 (−3.8%) |
| BMI | 34.3 | 33 | −1.3 (−3.7%) |
| Fat Mass (lbs.) | 77.7 | 72.1 | −5.6 (−7.2%) |
| Visceral Fat Rating | 13.8 | 12.8 | −1.0 (−7.2%) |
| Waist (cm) | 113.6 | 106.7 | −6.9 (−6.1%) |
| Hips (cm) | 115 | 111 | −4.0 (−3.5%) |
| Waist/Hip Ratio | 1.77 | 1.66 | −0.1 (−6.1%) |
| Stress/Anxiety Score | 10.5 | 5.3 | −5.1 (−49.0%) |

Changes from baseline visit 1 to visit 4 in markers of glycemic control and insulin sensitivity are listed in Table 8. Levels of Hemoglobin A1C (−9.2%), a measurement of DM control over a 3-month period improved while fasting blood glucose improvement was even more pronounced (−17.9%). Markers of insulin resistance, an important cause of type 2 DM and other diseases, decreased greatly with insulin levels falling (−18.7%) and HOMA-IR, a standard calculation of insulin resistance, dropping even more extensively (−36.6%).

TABLE 8
CHANGES IN MARKERS OF GLYCEMIC
CONTROL AND INSULIN RESISTANCE

| | Visit 1 | Visit 4 | |
|---|---|---|---|
| | (Week 1) | (Week 13) | Change |
| Hemoglobin A1C | 7.5 | 6.8 | −0.7 (−9.2%) |
| Insulin Level | 15.8 | 12.8 | −3.0 (−18.7%) |
| (uIU/ml) | | | |
| Fasting Glucose | 153.7 | 126.3 | −27.5 (−17.9%) |
| (mg/dl) | | | |
| HOMA-IR | 6.5 | 4.1 | −2.4 (−36.6%) |

Changes from baseline visit 1 to visit 4 in total and LDL cholesterol levels were minimal but favorable in this group of subjects that generally were already being treated with statin medications. (Table 9) Improvements in HDL levels were more substantial (3.4%) as were improvements in triglyceride levels (−12.1%). Levels of hsCRP a marker of general inflammation and associated with cardiovascular risk were reduced (−2.8%).

TABLE 9
CHANGES IN LIPID PANEL

| | Visit 1 | Visit 4 | |
|---|---|---|---|
| | (Week 1) | (Week 13) | Change |
| Total Cholesterol | 179.1 | 177.2 | −1.9 (−1.0%) |
| (mg/dl) | | | |
| LDL Cholesterol | 100.5 | 99.8 | −0.7 (−0.7%) |
| (mg/dl) | | | |
| HDL Cholesterol | 43.7 | 45.2 | 1.5 (3.4%) |
| (mg/dl) | | | |
| Triglycerides | 183.1 | 161.0 | −22.1 (−12.1%) |
| hsCRP | 5.5 | 5.3 | −0.2 (−2.8%) |

Discussion: Among the 15 subjects completing this study, weight loss was demonstrated and averaged 7.9 pounds over 13 weeks. This weight loss is impressive because treatments for type 2 DM often are associated with weight gain. One explanation for this usual weight gain is that as blood sugar control is improved with intervention, less glucose is generally lost through renal oversaturation (glycosuria), retaining these calories, and weight gain is often seen initially with diabetic treatments. Generally, any weight loss achieved in the first 3 months of diabetes treatment is considered important. In addition, the preferential loss of fat mass as demonstrated in this study is particularly desirable in treating type 2 DM.

Improvements in glycemic control and insulin resistance were the most impressive results of the study. Hemoglobin A1C and fasting blood glucose over 13 weeks compares favorably with traditional oral anti-diabetic treatments like metformin or pioglitazone. While the initial average A1C at baseline (7.5) indicated poor diabetic control, the average A1C level at V4 (6.8) met the goal A1C level recommended for diabetic patients (less than 7.0). Changes in fasting insulin level and HOMA-IR actually exceeded that seen with these prescribed medications. The large improvements in insulin resistance demonstrated by the GWMP system with the Release supplement suggest a beneficial role in other disease states including the Metabolic Syndrome.

Favorable changes to other laboratory tests were observed from baseline visit 1 to visit 4 and are listed in Table 10. Improvement in liver transaminase enzymes (AST and ALT) are often seen with weight loss and most likely reflect decreased inflammation from fatty liver. Changes in sex hormones are also seen following weight loss and represent decrease peripheral fat conversion of hormone pre-cursers and suggest a benefit in patients with Polycystic Ovary Disease (PCOS). No other significant changes in metabolic panel values, PSA or other safety variables were observed. Three subjects terminated from the study due to gastrointestinal adverse events including loose bowel movements or abdominal cramps. No serious adverse events were identified.

TABLE 10
CHANGES IN LIVER TRANSAMINASES AND SEX HORMONES

| | Visit 1 (Week 1) | Visit 4 (Week 13) | Change |
|---|---|---|---|
| AST (mg/dl) | 28.2 | 24.7 | −3.5 (−12.3%) |
| ALT (mg/dl) | 34.4 | 29.9 | −4.5 (−13.0%) |
| Progesterone | 1.1 | .3 | −0.8 (−76.9%) |
| (women) | | | |
| Estradiol | 34.4 | 24.8 | −9.6 (−27.9%) |
| (women) | | | |

The Stress/Anxiety questionnaire was a 20-question self-administered written test that served as a general marker of psychological health. Although the study was not statistically powered for this endpoint, the change in average score on this questionnaire was impressive (−49%) and suggest the opportunity for further study.

The study was limited by selection of subjects at only one clinical site. In addition, the study was open-label and lacks the rigor of a double-blinded placebo-controlled study. In addition, poor subject compliance with the GWMP diet and exercise component may also have influenced the results of the study. Although compliance with the Release supplement as obtained by pill counts (95 percent overall compliance by pill count) was excellent, compliance with diet and exercise recommendations was variable and more difficult to quantify. The relatively high drop-out rate (10/25-40%) of the subjects consented for the study reflect this difficulty in obtaining compliance with the program. Frustration with previous diet plans and unrealistic expectations about lifestyle change likely contributed to drop outs. In addition, enrolling a population with type 2 diabetes that likely has failed other attempts at diet, exercise and medical therapy presented challenges to compliance.

In summary, the GOLO Weight Management Program with the Release supplement demonstrated weight loss and improvement in glycemic control comparable to standard prescription anti-diabetic medications in this small, single center study. Improvements in markers of insulin resistance were impressive and exceeded those seen by existing anti-diabetic medications including the Gold Standard for treatment of insulin resistance pioglitazone. Further studies will be needed to evaluate the role of the GOLO Weight Management Program with the Release supplement in diabetic and non-diabetic populations.

### Example 2

### Efficacy of a Diet Program on Body Weight in Overweight and Obese South Africans

### Background

Over the course of several years the Medical Nutritional Institute (MNI) based in Johannesburg South Africa developed a weight loss program to help combat obesity and weight related conditions. The program included a lifestyle change plan, meal plan and dietary supplement labeled as Antagolin.

Five studies were conducted in South Africa, in Cape Town and Johannesburg during 2009-2014 (Table 11). The studies are referred to by the names SANLAM, ABSA, SAMANCOR, X-STRATA, and LIGHTHOUSE.

NaturPro Scientific LLC reviewed all study data provided and evaluated the apparent integrity and completeness of the data, and conducted statistical analysis, and wrote the reports. Data for each study was analyzed independently and then in a pooled analysis that combined study data of the same duration (12 or 25-27 weeks). All data from subjects completing the study were analyzed based on changes in endpoints from baseline using means, standard deviation and single-factor ANOVA (Microsoft Excel).

### Methods

Participants for these studies were recruited through their employer as part of a wellness initiative. With the exception of ABSA which was 12 weeks only, all studies were designed as 12-weeks in duration, with the option given to subjects to continue the study for an additional 13 weeks.

The study subjects participated in one of two treatment groups: the Complete Program, or the Program without the dietary supplement, which was named the “Control Program”.

TABLE 11
SUMMARY OF NUMBER OF SUBJECTS AND LENGTH
OF STUDIES.
South Africa Study Summary

| | Length | Subjects | | |
|---|---|---|---|---|
| Study | Weeks | (N) | Treatment | Year |
| SANLAM | 12 | 17 | Complete Program | 2009 |
| (12 wks) | 12 | 4 | Control-Program Only | |
| SANLAM | 25 | 17 | Complete Program | |
| (Ext 25 wks) | 25 | 4 | Control-Program Only | |
| SANLAM | 61 | (subset of | Complete Program | |
| (Ext 61 wks) | | 8 obese) | | |
| SANLAM | 102 | (subset of | Complete Program | |
| (Ext 102 wks) | | 6 obese) | | |
| ABSA | 12 | 13 | Complete Program | 2010 |
| | 12 | 5 | Control-Program Only | |
| SAMANCOR | 12 | 14 | Complete Program | 2011 |
| | 12 | 6 | Control-Program Only | |
| SAMANCOR | 27 | (subset of 10) | Complete Program | |
| (Ext 27 wks) | | | | |
| X-STRATA | 12 | 22 | Control-Program Only | 2012 |
| | 29 | 22 | Control-Program Only | |
| LIGHTHOUSE | 12 | 10 | Complete Program | 2014 |
| | 16 | 10 | Complete Program | |
| Pooled 12 Week | 12 | S4 | Complete Program | |
| Results | 12 | 37 | Control-Program Only | |
| Pooled 25 Week | 25-27 | 27 | Complete Program | |
| Results | 25-29 | 26 | Control-Program Only | |

### Intervention

The Program portion of the system included tools for achieving a healthy lifestyle which included an eating plan based on portion control and caloric restriction, behavior modification guidance in the form of booklets that included a self-assessment and an exercise guide promoting strength and circuit training exercise to support cardiovascular and muscle maintenance and development.

The supplement contained a blend of plant-derived ingredients and minerals consisting of banaba leaf, barberry bark extract, inositol, and chromium. Recommended dosage was 2 capsules twice a day.

The Program including the supplement was termed the “Complete Program”, and the Program without the supplement was named the “Control Program”.

### Inclusion and Exclusion Criteria

Adults older than 20 years old, with a body mass index >25 kg/m² were included in all studies. Subjects were required to be healthy, pre-diabetic or diabetic people measured as overweight or obese. Excluded were pregnant or lactating subjects, and individuals with known allergies to the dietary supplement or any of its ingredients, determined by questionnaire. Subjects were instructed to continue to take all medications that they were prescribed before entering the study. Any subsequent changes in medication were made under supervision of the subjects' personal physicians.

### Study Endpoints

In all studies, food and exercise logs were recorded by subjects daily, and clinical and anthropometric measurements such as vital signs and body weight were measured by full time staff members of the company conducting the trial. Each had a recognized medical qualification, including registered nurses, a dietician, a biokineticist and a pharmacist.

### Compliance and Follow-Up

Compliance was based on level of adherence to diet, supplementation and exercise recommendations as well as appearance for study visits. A minimum average compliance of 50% per subject was required for inclusion in the data analysis. After 26 weeks, obese subjects were invited to continue on the program for an additional 26 weeks.

### Ethical Requirements

For all studies conducted, informed consent was given by all subjects. Study subjects were not compensated for participation.

### Results

Overall, 54 subjects completed 12 weeks on the Complete Program, and 27 subjects continued for 25-27 weeks (See Table 11). 37 subjects on the control program completed 12 weeks, and 26 subjects on the control program completed 25-29 weeks. Of eight dropouts, none were due to study- or treatment-related issues. Table 23 below provides the reasons for all dropouts.

In the SANLAM study, an average of 32.1 and 53.4 pounds were lost after 12 and 26 weeks, respectively, in those subjects using the Complete Program (Table 13).

TABLE 12
SOUTH AFRICA STUDY SUMMARY AVERAGE STUDY RESULTS
IN INDIVIDUAL STUDIES AND IN POOLED RESULTS—SANLAM STUDY

| | Length | | | Weight Loss | Weekly | Waist Loss |
|---|---|---|---|---|---|---|
| Study | Weeks | Subjects (N) | Treatment | (lb) | Loss (lb) | (inches) |
| SANLAM (12 wks) | 12 | 17 | Complete Program | 32.1 | 2.7 | * |
| | 12 | 4 | Control-Program Only | 8.9 | 0.7 | * |
| SANLAM (Ext 25 wks) | 25 | 17 | Complete Program | 53.4 | 2.1 | 6.8 |
| | 25 | 4 | Control-Program Only | 9.5 | 0.4 | 6.3 |
| SANLAM (Ext 61 wks) | 61 | (subset of 8 obese) | Complete Program | 62.0 | 1.0 | 10.4 |
| SANLAM (Ext 102 wks) | 102 | (subset of 6 obese) | Complete Program | 83.8 | 0.8 | 12.1 |
| ABSA | 12 | 13 | Complete Program | 16.7 | 1.4 | 3.8 |
| | 12 | 5 | Control-Program Only | 7.7 | 0.6 | 8.7 |
| SAMANCOR | 12 | 14 | Complete Program | 20.5 | 1.7 | 2.2 |
| | 12 | 6 | Control-Program Only | 5.5 | 0.5 | 2.2 |
| SAMANCOR (Ext 27 wks) | 27 | (subset of 10) | Complete Program | 32.6 | 1.2 | 4.7 |
| X-STRATA | 12 | 22 | Control-Program Only | 6.7 | 0.6 | * |
| | 29 | 22 | Control-Program Only | 10.4 | 0.4 | 2.7 |
| LIGHTHOUSE | 12 | 10 | Complete Program | 9.0 | 0.8 | * |
| | 16 | 10 | Complete Program | 21.6 | 1.4 | 5.0 |
| Pooled 12 Week | 12 | 54 | Complete Program | 24.0 | 2.0 | * |
| Results | 12 | 37 | Control-Program Only | 8.1 | 0.7 | * |
| Pooled 25 Week | 25-27 | 27 | Complete Program | 44.9 | 1.7 | 6.8 |
| Results | 25-29 | 26 | Control-Program Only | 8.9 | 0.3 | 3.4 |

* not measured

TABLE 13
AVERAGE BODY WEIGHT FOR SUBJECTS IN THE SANLAM
STUDY ON THE COMPLETE PROGRAM.

| SANLAM (n = 17) | Baseline | 12 weeks | 26 weeks |
|---|---|---|---|
| Weight (lb) | 270.9 | 238.8 | 217.4 |
| p-value | | 0.08 | 0.0041 |

A subset of 8 obese subjects in SANLAM who began the study weighing more than 240 pounds and who completed the initial 25 weeks continued the Complete Program for an additional 36 weeks (61 weeks total) experienced an average weight loss of 62 pounds (Table 12). The difference from baseline trended to significance (p<0.059). Six of these subjects continued for an additional 41 weeks (102 weeks total), and this group experienced an average weight loss of 83 pounds (p=0.03).

A trend for significant reduction in body weight (lb) (p=0.059) was observed for 8 obese subjects in SANLAM (arbitrarily coded A-H) who continued on the trial for more than one year (61 weeks).

TABLE 14
SANLAM RESULTS IN OBESE SUBJECTS USING THE
COMPLETE PROGRAM.

| Subject | Baseline | 1 year | Change (lbs) |
|---|---|---|---|
| A | 426 | 354 | −72 |
| B | 252 | 209 | −43 |
| C | 242 | 176 | −65 |
| D | 315 | 252 | −63 |
| E | 255 | 176 | −79 |
| F | 321 | 255 | −67 |
| G | 251 | 224 | −27 |
| H | 267 | 184 | −83 |
| AVG | 291 | 229 | −62 |

### ABSA Study

Thirteen subjects on the Complete Program entered and completed the 12-week ABSA study. An average reduction in weight of 16.7 pounds was observed, although this result was not significant (p=0.46).

TABLE 15
AVERAGE BODY WEIGHT FROM ABSA IN SUBJECTS
ON THE COMPLETE PROGRAM.

| ABSA (n = 13) | Baseline | 12 weeks |
|---|---|---|
| Weight (lb) | 229.0 | 212.3 |
| p-value | | 0.46 |

A subset of four subjects in the ABSA study on the Complete Program continued for 43 weeks. An average weight loss of 36.6 lb was observed in these subjects, and the result was significant despite the small sample size (p=0.05).

TABLE 16
AVERAGE BODY WEIGHT FROM ABSA FOR SUBJECTS
CONTINUING ON THE STUDY FOR 43 WEEKS.

| ABSA (n = 4) | Baseline | 43 weeks |
|---|---|---|
| Weight (lb) | 186.1 | 149.5 |
| p-value | | 0.05 |

### SAMANCOR Study

In the SAMANCOR study, 14 subjects using the Complete Program lost an average of 20.5 pounds, a result that was not significant due to the small sample size.

TABLE 17
AVERAGE BODY WEIGHT FROM SAMANCOR IN
SUBJECTS ON THE COMPLETE PROGRAM.

| SAMANCOR (n = 14) | Baseline | 12 weeks |
|---|---|---|
| Weight (lb) | 239.4 | 218.9 |
| p-value | | 0.20 |

A subset of ten subjects using the Complete Program continued for 27 weeks in the SAMANCOR study, resulting in an average weight loss of 32.6 pounds, a significant result (p=0.001).

TABLE 18
AVERAGE BODY WEIGHT FROM SAMANCOR FOR
SUBJECTS USING THE COMPLETE PROGRAM.

| SAMANCOR (n = 10) | Baseline | 27 weeks |
|---|---|---|
| Weight (lb) | 229.7 | 197.1 |
| p-value | | 0.001 |

### Lighthouse Study

In the LIGHTHOUSE study, an average of 19.9 pounds were lost over 12 weeks, a result which was not significant (p=0.38).

TABLE 19
AVERAGE BODY WEIGHT FROM LIGHTHOUSE FOR
SUBJECTS USING THE COMPLETE PROGRAM.

| LIGHTHOUSE (n = 10) | Baseline | 12 weeks |
|---|---|---|
| Weight (lb) | 223.8 | 203.9 |
| p-value | | 0.38 |

### Pooled Results

A pooled analysis was conducted on studies with the same durations and study design in order to understand overall population effects. Pooled subjects completing 12 weeks on the Complete Program lost an average of 24.0 pounds, an effect which was significant from baseline (p=0.02). Subjects completing 25 weeks on the Complete Program lost an average of 44.9 pounds, which was also significant (p=0.0004).

TABLE 20
POOLED RESULTS OF AVERAGE BODY WEIGHT FOR SUBJECTS
COMPLETING 12- AND 25-WEEK STUDIES.

| | Subjects | | Length | Weight Loss | Weekly | Waist Loss |
|---|---|---|---|---|---|---|
| Study | (N) | Treatment | Weeks | (lb) | Loss (lb) | (inches) |
| Pooled | 54 | Complete GOLO Program | 12 | 24.0 | 2.0 | * |
| Pooled | 37 | Control-Program Only | 12 | 8.1 | 0.7 | * |
| Pooled | 27 | Complete GOLO Program | 25-27 | 44.9 | 1.7 | 6.8 |
| Pooled | 26 | Control-Program Only | 25-29 | 8.9 | 0.3 | 3.4 |

(*not measured).

The changes in body weight for control subjects pooled across the 12- and 25-week studies were similar to those previously published on other widely available diet programs. Although subjects in the control group lost an average of 8.1 and 8.9 pounds over 12 and 25 weeks, respectively, the effect was not significant (p>0.05).

Below, individual pooled 12-week data is shown.

TABLE 21
INDIVIDUAL RESULTS FOR AVERAGE BODY WEIGHT FOR
SUBJECTS ON THE COMPLETE GOLO PROGRAM, POOLED
FOR SUBJECTS COMPLETING 12 WEEKS.
Complete Program
Pooled 12-weeks

| Weight (lb) | |
|---|---|
| Baseline | 12 weeks |
| 255.3 | 211.2 |
| 245.6 | 195.8 |
| 267.2 | 224.2 |
| 252.0 | 218.5 |
| 321.2 | 280.2 |
| 201.1 | 173.9 |
| 425.9 | 369.3 |
| 270.3 | 243.2 |
| 241.6 | 218.9 |
| 245.1 | 219.8 |
| 240.5 | 222.2 |
| 360.9 | 282.4 |
| 251.3 | 232.8 |
| 315.3 | 284.8 |
| 239.9 | 221.3 |
| 208.3 | 176.4 |
| 263.0 | 239.4 |
| 203.0 | 186.5 |
| 240.1 | 210.5 |
| 214.1 | 197.2 |
| 242.5 | 220.3 |
| 253.1 | 218.7 |
| 199.5 | 191.8 |
| 255.3 | 237.9 |
| 254.9 | 229.7 |
| 227.7 | 204.4 |
| 207.0 | 181.0 |
| 197.1 | 179.7 |
| 249.1 | 227.5 |
| 368.2 | 339.5 |
| 240.3 | 238.3 |
| 249.1 | 228.6 |
| 205.8 | 184.3 |
| 295.9 | 263.0 |
| 219.6 | 203.5 |
| 175.0 | 154.2 |
| 228.0 | 208.6 |
| 303.8 | 294.3 |
| 165.8 | 147.3 |
| 233.7 | 210.1 |
| 160.2 | 145.1 |
| 212.1 | 189.2 |
| 173.1 | 150.4 |
| 177.0 | 158.3 |
| 257.3 | 244.5 |
| 200.6 | 189.8 |
| 297.6 | 282.2 |
| 197.1 | 183.0 |
| 140.4 | 130.3 |
| 297.4 | 292.3 |
| 280.9 | 245.8 |
| 181.9 | 153.4 |
| 249.8 | 230.6 |
| 311.3 | 309.5 |
| 243.9 | 219.9 |
| | Average |

Below, individual pooled 25-week data is shown.

TABLE 22
INDIVIDUAL RESULTS FOR AVERAGE BODY WEIGHT FOR
SUBJECTS ON THE COMPLETE PROGRAM, POOLED FOR
SUBJECTS COMPLETING 25 WEEKS.
Complete Program
Pooled 25-weeks

| Weight (lb) | |
|---|---|
| Baseline | 25 weeks |
| 203.0 | 188.3 |
| 227.7 | 197.1 |
| 240.1 | 196.1 |
| 214.1 | 186.3 |
| 253.1 | 207.2 |
| 242.5 | 202.8 |
| 199.5 | 184.1 |
| 255.3 | 228.0 |
| 254.9 | 222.7 |
| 207.0 | 179.7 |
| 360.9 | 229.5 |
| 255.3 | 178.8 |
| 245.6 | 172.6 |
| 267.2 | 189.8 |
| 252.0 | 200.2 |
| 321.2 | 255.5 |
| 201.1 | 160.3 |
| 425.9 | 341.9 |
| 270.3 | 224.9 |
| 239.9 | 201.1 |
| 241.6 | 205.9 |
| 251.3 | 216.0 |
| 208.3 | 181.7 |
| 315.3 | 275.1 |
| 245.1 | 216.0 |
| 240.5 | 212.5 |
| 263.0 | 235.0 |
| 255.6 | 210.7 |
| | Average |

TABLE 23
WEIGHTS FOR SUBJECTS WITHDRAWING FROM THE STUDIES,
AND REASON FOR WITHDRAWAL.

| | Duration | Weight (lb) | | |
|---|---|---|---|---|
| Study | (weeks) | Baseline | End | Reason cited |
| SAN LAM | 14 | 259.9 | 257.5 | became |
| | | | | pregnant |
| ABSA | 4 | 151.5 | 149.5 | work stress; |
| | | | | failed to attend visits |
| ABSA | 8 | 179.7 | 172.4 | work stress; |
| | | | | failed to attend visits |
| SAMANCOR | 8 | 210.1 | 203.3 | alcoholism |
| SAMANCOR | 4 | 419.8 | 399.3 | Work stress |
| X-STRATA | 0 | 305.1 | NA | failed to attend visits |
| X-STRATA | 4 | 219.1 | 207.5 | lost interest |
| X-STRATA | 8 | 331.8 | 289.0 | employment |
| | | | | change |

### Example 3

### Efficacy of a Diet Program on Body Weight in Overweight Americans

### Introduction

The NIH reports that 68.8% of adults in the USA are considered to be overweight or obese (35.7% obese and 6.3% have extreme obesity). It is generally accepted that being overweight increases risk factors for chronic disease. According to the Centers for Disease Control, 29.1 million Americans live with type 2 diabetes and 86 million with pre-diabetes. According to the U.S. National Institutes of Health, including the National Institute of Diabetes and Digestive and Kidney Diseases (NIDDK), high body weight and inadequate physical activity are two primary causes of insulin resistance and pre-diabetes. For many overweight people, changing dietary habits is key to maintaining a healthy body weight.

This report summarizes a pilot study performed in the USA to evaluate the safety and efficacy of a weight loss system originally developed and tested in South Africa by physicians at the Medical Nutritional Institute (MNI) in Johannesburg, South Africa, to provide a more effective weight loss solution.

### BACKGROUND

MNI was started in 2002 to focus on weight management and healthcare training services. MNI developed the weight loss system to address previous limitations of corporate wellness programs to deliver sustainable weight loss and measurable health benefits. Conrad Smith M.D. and Mariaan Du Plessis (registered pharmacist) developed an approach to improve insulin management during weight loss programs. MNI developed a 3-tiered solution centered on insulin management in conjunction with caloric control and intervention with a plant and mineral based dietary supplement, containing ingredients researched to address insulin performance. MNI implemented two studies within corporate wellness programs in 2009 and 2010 and an additional three studies from 2011 through 2014.

In 2010, MNI partnered with GOLO, LLC in the USA (Newark, Del.), and GOLO implemented a pilot study in 2011 to understand whether the initial results in South Africa could be replicated in a separate population of subjects.

### Methods

A 26-week, open-label pilot study of 35 overweight/obese human subjects was conducted in Delaware in the United States by the marketer GOLO to determine the efficacy of a weight loss program “GOLO for Life”. The study was conducted in the U.S. in 2010-2011 and recruited 49 subjects. Study participants were recruited through local newspaper ads and local postings. The study was comprised of weekly study visits with key data collected at baseline, 30 days, 90 days, and 26 weeks (Study 1), with an option for subjects to continue with the study for an additional 26 weeks (Study 1a). At study completion after 26 weeks, 23 subjects opted to continue on the program as a maintenance study for an additional 26 weeks. Each subject's baseline values were recorded and served as the basis by which the program's efficacy was measured.

TABLE 24
OVERVIEW OF STUDIES ON DIET PROGRAM.

| Study# | Location | N* | Weeks |
|---|---|---|---|
| 1 | United States | 35 | 26 |
| 1a | United States | 23 | 52 |

*N is the number of subjects completing the study. Study #1a is the continuation of study #1 after an additional 26-week follow-up.

### The Diet Program

The diet program included behavior modification guidance in the form of booklets that included a self-assessment, an eating plan based on food groups, portion control and caloric restriction, and a dietary supplement as three capsules, three times per day. The supplement contained a blend of plant-derived ingredients and minerals consisting of banana leaf, barberry bark extract, apple fruit extract, salacia bark extract, gardenia fruit extract, rhodiola root, inositol, chromium, zinc and magnesium. The diet program used in this study may be best categorized as self-directed, with counseling and support available online, by email communications, or through a toll-free phone number. Subjects were provided with written handouts for the meal plan that included guidance on serving recommendations from the major food groups, with portion control and a self-assessment behavioral handout. Subjects were also provided with food and exercise logs. Subjects were directed to attempt 15 minutes of exercise per day or 105 minutes per week and to preferably exercise using high intensity workouts (HIT) such as walking with 30 second bursts and 30 second rest periods. Optional exercise classes were made available to the subjects twice per week.

### Study Criteria

Adults older than 20 years old, with a body mass index>25 kg/m.sup.2 were included in the study. Subjects were required to be healthy overweight or obese. Excluded were type 2 diabetics, pregnant or lactating subjects, determined by questionnaire, and individuals with known allergies to the dietary supplement or any of its ingredients. Subjects were instructed to continue to take all medications that they were taking at the time that the study began. Any subsequent changes in medication were made under supervision of the subjects' personal physician. Subjects were not compensated to join the trial or for participating in the trial.

Subjects were requested to visit the study center weekly with a minimum monthly visit to remain in the study. At each visit, body weight, body mass index (BMI), visceral fat, body fat, muscle loss and metabolic age as measured by a Tanita Body Composition Analyzer SC-331S were measured.

Body circumference measurements were taken at the waist, shoulders, chest, bicep, hips and thighs, and blood pressure (mmHg) and heart rate were also measured. Subjects provided completed food and exercise logs at the visit and their feedback on the Program. Subjects self-reported changes in dress and pants sizes. Front and side pictures of all subjects were taken at baseline and various intervals and at end points to verify results. Medication history and reduction or elimination of medications was also recorded.

At baseline, 3 months, and 6 months, blood work was taken and fasting blood glucose (mg/dL), HbAlc (% DCCT) and blood lipids (triglycerides, total cholesterol, LDL, HDL) were measured using LabCorp or equivalent clinical chemistry providers.

### Compliance

Compliance was measured at each weekly visit and at least monthly through exercise logs and food logs kept by subjects, and a count of supplement capsules remaining during each study visit. A minimum average compliance of 50% was required for inclusion in the data analysis. Compliance was based on level of adherence to diet, supplementation and exercise recommendations.

### Ethical Requirements

Informed consent was obtained for all subjects. Study subjects were not compensated for participation. No institutional review board was used.

### Data Analysis

Blake Ebersole (NaturPro Scientific LLC, Carmel, Ind.) reviewed all data provided by GOLO, LLC, evaluated the apparent integrity and completeness of the data, conducted statistical analysis, and wrote the report. All data from subjects completing the study were analyzed based on changes in endpoints from baseline using means, standard deviation and single-factor ANOVA using Microsoft Excel.

### Results

Out of 49 subjects entering the study, 35 completed the full 26 week study. Of the 14 subjects who left the study, none were due to adverse events (Table 21). Data from dropouts were not included in the analysis of group results if they did not reach the 26 week mark and complete blood work. All drop out subjects lost weight, with data and reason for dropping out stated in Table 26 below.

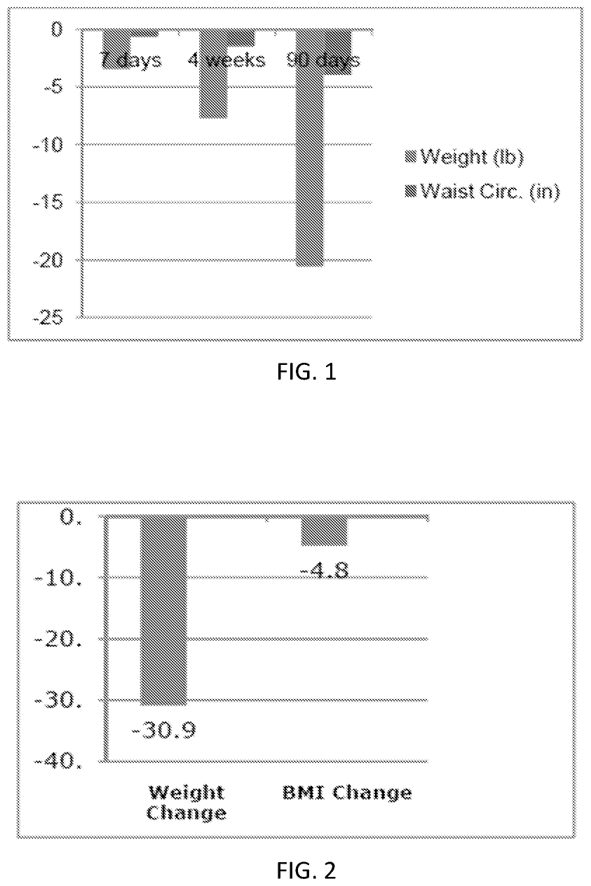

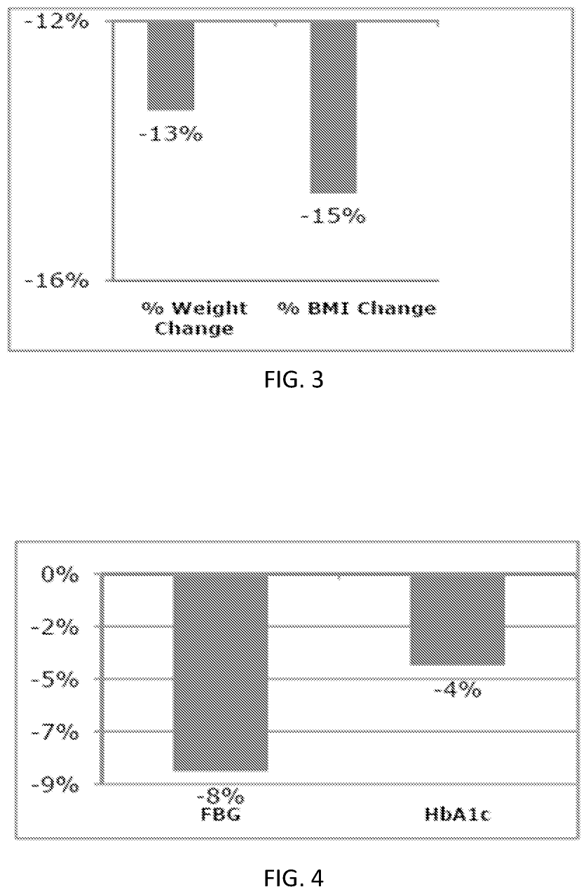

After 7 days, body weight in 34 subjects was reduced by an average of 3.4 lbs., and after 30 days, an average of 7.7 lbs. weight loss was observed (FIG. 1). One subject did not report data at 7 days and 30 days. After 90 days, average body weight was reduced by 20 pounds, and waist circumference was reduced by an average of four inches. After 26 weeks, the average weight loss was 30.9 pounds, which was significant from baseline (p<0.005) (FIG. 2). The average decrease in BMI at 26 weeks was 4.8 units (FIG. 3).

Average waist circumference, pants and dress size, fat percentage, total cholesterol, and triglycerides all decreased significantly from baseline after 26 weeks (Table 28). Muscle loss did not significantly decrease, suggesting the diet program reduced body fat without significant loss of muscle mass. For the subjects who remained in the study for an additional 26 weeks, all measures continued to be a significant reduction from baseline, except for total cholesterol.

TABLE 25
SUMMARY OF DATA IN THE U.S. DIET STUDY, BASED ON
AVERAGE VALUES.
VALUES IN RED WERE NOT SIGNIFICANT (P > 0.05).

| | Baseline | 90 Day | 26 Weeks (n = 35) | 1 Year (n = 23) |
|---|---|---|---|---|
| Weight | 210.8 | 191.4 | 178.8 | 167.8 |
| | | −19.3 ± 8.55 | −30.9 ± 11.1 | −36.6 ± 14.7 |
| (lbs) | | [symbol] | (p = 0.0024) | (p = 0.000049) |
| Waist | 42.1 | 39.0 | 35.7 | 34.5 |
| Circum- | | 3.09 ± 1.70 | −5.48 ± 1.75 | −6.94 ± 2.52 |
| ference | | (p = 0.03) | (p = 0.000031) | (p = 0.0000016) |
| (in) | | | | |
| BMI | 32.9 | | 28.1 | 25.9 |
| | | | -4.8 ± 1.8 | −6.1 ± 3.0 |
| | | | (p = 0.00064) | (p = 0.0000025) |
| Dress Size | 16.2 | | 9.33 | 7.3 |
| | | | −7 ± 2 | −8 ± 2 |
| | | | (p = 0.000024) | (p = 0.0000049) |
| Pants Sizes | 40.7 | | 36.5 | 33.5 |
| (waist | | | −5.27 ± 1.01 | −7.6 ± 2.6 |
| inches) | | | (p = 0.00025) | (p = 0.00078) |
| Muscle | 129.7 | | 122.7 | 127.3 |
| (lbs) | | | −7.1 ± 5.1 | −2.3 ± 7.0 |
| | | | (p = 0.24) | (p = 0.78) |
| % Muscle | 62.30% | | 69.20% | 75.90% |
| | | | 6.9% ± 4.3% | 12.5% ± 4.6% |
| | | | (p = 0.00067) | (p = 0.0000017) |
| Fat (lbs) | 80.0 | | 56.2 | 40.4 |
| | | | −23.8 ± 11.7 | −34.3 ± 12.6 |
| | | | (p = 0.00074) | (p = 0.000000017) |
| % fat | 37.00% | | 30.80% | 24.10% |
| | | | −6.9% ± 4.3% | −12.5 ± 4.6% |
| | | | (p = 0.00068) | (p = 0.0000017) |
| Cho- | 184.5 | | 166.9 | 174.7 |
| lesterol | | | −17.6 ± 33.9 | −7 ± 35 |
| | | | (p = 0.041) | (p = [symbol] ) |
| LDL | 106.5 | | 91.5 | 94.4 |
| | | | −13.9 ± 29.1 | −8 ± 31 |
| | | | (p = 0.68) | (p = 0.38) |
| HDL | 57.4 | | 60.5 | 64.8 |
| | | | 3.11 ± 9.93 | 9 ± 16 |
| | | | (p = 0.41) | (p = 0.064) |
| Tri- | 108.3 | | 74.4 | 76.9 |
| glyceride | | | −33.9 ± 48.1 | −37 ± 55 |
| | | | (p = 0.0037) | (p = 0.0067) |
| CHL/HDL | 3.54 | | 2.88 | 2.88 |
| ratio | | | −0.63 ± 0.90 | −0.64 ± 1.23 |
| | | | (p = 0.020) | (p = 0.07) |
| HDL/LDL | 2.09 | | 1.51 | 1.61 |
| ratio | | | −0.44 ± 0.70 | −0.42 ± 0.98 |
| | | | (p = 0.035) | (p = 0.18) |

[symbol]

indicates data missing or illegible when filed

According to the NIDDK, fasting blood glucose and hemoglobin Ale (HbAlc) are primary indicators of blood sugar health and insulin resistance. At 26 weeks fasting blood glucose was reduced by an average of 8.5% (p<0.01) and HbAlc was reduced by an average of 3.9% (5.79 to 5.56, p=0.011) (FIG. 4). In a subset of 24 healthy pre-diabetic subjects with baseline HbAlc levels between 5.7 and 7.1, an average 6.9% decrease was found at 90 days (p<0.0001). A 4% average reduction was observed after 26 weeks, which was significant (p=0.002). At 52 weeks, an average 7.3% decrease was found in these subjects (p<0.001).

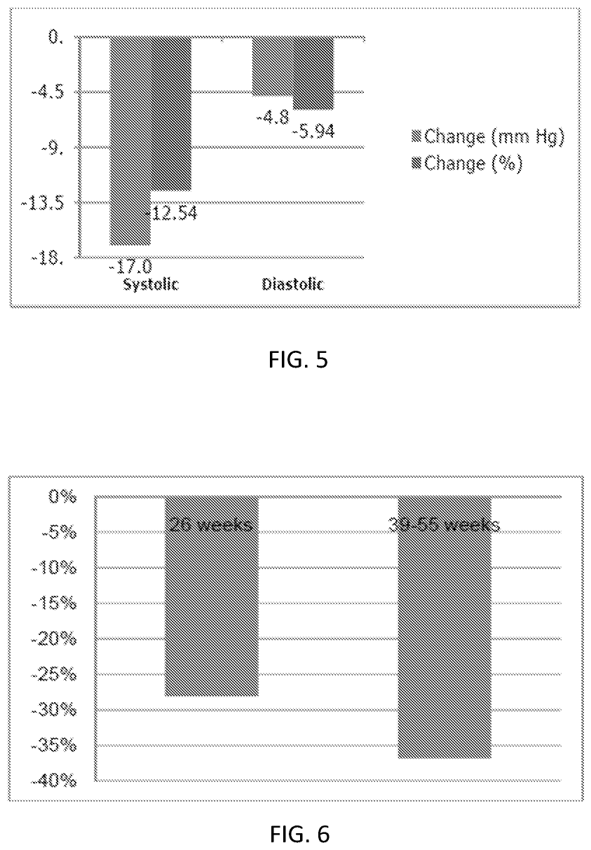

In all subjects, average systolic and diastolic blood pressure was reduced by 12.5% (p<0.001) and 6.0% (p<0.05), respectively after 26 weeks (FIG. 5).

Visceral fat was significantly reduced over 26 weeks by an average of 28% (from 10.00% to 7.19%, p=0.005, FIG. 6) and by an average of 37% in subjects who continued for 39-55 weeks (from 10.00% to 6.32%, p<0.0001). Of the 35 subjects, 11 had a visceral fat percentage greater than 13% at the start (average 15.82%). At 26 weeks, the average visceral fat percentage for these 11 subjects was 11.45%.

Metabolic age, calculated from body composition analysis, also decreased from an average of 56.3 to 43.2 years of age (p<0.0001) at 26 weeks. Although metabolic age is an estimated value, it can be used as supportive data with respect to the number of other improvements observed in this study on primary biomarkers such as body weight.

Fourteen (14) of the USA study participants had been prescribed a total of 35 medications at the start of the study. After 26 weeks, more than half (18) prescription medications were eliminated, and 6 were prescribed at a lower dosage, all under the supervision of the subjects' personal physician. 14 subjects left the study before 26 weeks due to various factors (Table 26). No dropouts were due to adverse events. All dropouts showed a reduction in weight.

TABLE 26
BODY WEIGHT CHANGES FROM SUBJECTS WHO
DISCONTINUED THE 26-WEEK STUDY.

| | | | Weeks | |
|---|---|---|---|---|
| Baseline (lb) | End (lb) | Difference (lb) | in Study | Reasons |
| 227 | 212.6 | −14.4 | 16 | Family issues |
| 205 | 191.6 | −13.4 | 12 | Unknown |
| 173.4 | 165.6 | −7.8 | 10 | Moved away |
| 225.8 | 221.6 | −4.2 | 9 | Husband made her stop |
| 140.4 | 135.6 | −4.8 | 7 | Work |
| 240.4 | 229.6 | −10.8 | 5 | Drug problem |
| 210.4 | 199.8 | −10.6 | 5 | Mother sick |
| 274.6 | 268.2 | −6.4 | 8 | Health issues |
| 168.8 | 155.6 | −13.2 | 14 | Travelling time |
| 151.2 | 123.2 | −28 | 14 | Time issues |
| 184.6 | 177.2 | −7.4 | 7 | Scheduling conflicts |
| 165.4 | 145.2 | −20.2 | 20 | Time issues |
| 151.4 | 139.6 | −11.8 | 15 | Pregnancy |
| 232.4 | 218 | −14.4 | 13 | Changed jobs-too far |

### Discussion

The diet program used in this study combines diet, exercise and nutritional supplementation together into a self-directed program. A number of studies have been performed using similar diet programs. A recent meta-analysis was performed that categorized randomized controlled human trials on diet programs into three categories: 1) “market leaders” such as Nutri-System® and Weight Watchers®, 2) very-low-calorie meal replacements, and 3) self-directed programs such as Atkins® and SlimFast®. Most of the diet plans studied combine face-to-face and group counseling, medical supervision, packaged foods or diet plans, and exercise recommendations.

The range of weight loss in 6-month studies on market leaders was between 3.6 and 8.1% of total body weight. Meanwhile, weight loss for very-low-calorie meal replacement programs ranged between 1.9 and 22% for study durations between 3 and 9 months, with most studies reporting an average reduction lower than 8%. For self-directed programs, studies ranging from 3-12 months demonstrated a range of average weight loss between 0 and 8.7%, with the predominant number of studies averaging less than 5% weight loss. The diet program used in this study may be best categorized as self-directed, with counseling and support available online, by email communications, or through a toll-free phone number. In this study, an average weight loss of 13% was observed in 26 weeks, which compares favorably to previous studies on self-directed programs. In addition to improvements in body weight and BMI, a number of blood markers associated with poor diet, cardiometabolic syndromes, and insulin resistance, including fasting blood sugar and HbAlc, were also improved compared to baseline values.

Glycated hemoglobin Ale (HbAlc) is a marker long used by physicians to determine a patient's average blood glucose levels over the previous 8-12 weeks. For this reason, HbAlc is generally considered a more reliable marker than fasting blood glucose to reflect average levels of blood glucose. Insulin resistance occurs when cells in the body are less responsive to insulin, which can lead to increases in blood sugar reflected in HbAlc and FBG levels. Thus, a reduction in HbAlc levels, along with improvements in body weight, together may indicate an improvement in insulin resistance. For example, changes in insulin resistance and HbAlc can be caused by exercise-mediated changes in body composition in older adults with type-2 diabetes. Other diet programs have also measured improvements in HbAlc and body weight. A 2016 study on Weight Watchers® showed a significant 6-month weight loss of 5.5%, and a significant decrease in HbAlc in pre-diabetic subjects following the program. A health coaching program in diabetics with a low socioeconomic status was also shown to reduce body weight, waist circumference and HbAlc.

The diet program used in the current study appeared to improve body weight favorably, consistent with previous studies on other similar diet programs. The diet program used in this study was affordable and did not require office visits, or food to be purchased, aside from an included dietary supplement requiring the consumption of three capsules per day. These factors could help to contribute to long-term compliance for a broader spectrum of people, as well as for those who have more weight to lose and need a long-term weight loss plan.

As with any study, dropouts and side effects can be a concern for weight loss plans. However dropouts from the studies all had reduced body weight at the time of withdrawal, and no withdrawals were due to treatment or study-related effects. Based on this early data, the diet program in this study may be an effective program that can be self-directed and also easily monitored by health coaches or physicians.

Although subjects were used as their own controls in this study, data from control groups in studies of other diet programs allow for some meaningful comparisons to be made. The amount and percentage of weight loss observed in this study was consistent with data reported by similar programs such as Weight Watchers and Nutrisystem. Despite the lack of a separate control group, an abundance of control data has been published. Further, the high degree of clinical relevance and supporting evidence on the individual elements of the intervention in this study adds a considerable degree of strength to the results observed. Larger studies are planned on the program to determine whether the effects observed can be repeated in other populations, and in larger sample sizes over longer durations.

Overall, subjects in this study showed significant reductions in body weight and BMI, and also experienced improvements in several markers of metabolic function. The data is consistent with previous results from the program, and with published data supporting the elements of the program. Thus, the effects observed in this study on a combination of dietary and behavioral interventions support the effectiveness of the GOLO for Life program for weight loss and several related clinical endpoints.

TABLE 27
INDIVIDUAL WEIGHT LOSS AFTER 26 WEEKS.

| Subject | Baseline | 26 weeks | Difference | |
|---|---|---|---|---|
| # | (lb) | (lb) | (lb) | Difference (%) |
| 1 | 205.8 | 174.6 | −31.2 | −15% |
| 2 | 220.8 | 189.2 | −31.6 | −14% |
| 3 | 238.0 | 203.6 | −34.4 | −14% |
| 4 | 205.0 | 189.0 | −16.0 | −8% |
| 5 | 155.6 | 143.0 | −12.6 | −8% |
| 6 | 177.8 | 152.6 | −25.2 | −14% |
| 7 | 238.0 | 185.2 | −52.8 | −22% |
| 8 | 192.6 | 156.0 | −36.6 | −19% |
| 9 | 246.0 | 206.0 | −40.0 | −16% |
| 10 | 220.2 | 184.4 | −35.8 | −16% |
| 11 | 156.8 | 135.8 | −21.0 | −13% |
| 12 | 197.0 | 153.4 | −43.6 | −22% |
| 13 | 182.6 | 152.6 | −30.0 | −16% |
| 14 | 213.8 | 183.0 | −30.8 | −14% |
| 15 | 233.0 | 190.6 | −42.4 | −18% |
| 16 | 182.6 | 163.6 | −19.0 | −10% |
| 17 | 375.4 | 332.4 | −43.0 | −11% |
| 18 | 188.0 | 162.8 | −25.2 | −13% |
| 19 | 201.4 | 161.8 | −39.6 | −20% |
| 20 | 250.2 | 221.6 | −28.6 | −11% |
| 21 | 173.2 | 144.0 | −29.2 | −17% |
| 22 | 172.0 | 157.2 | −14.8 | −9% |
| 23 | 183.2 | 152.8 | −30.4 | −17% |
| 24 | 206.4 | 160.4 | −46.0 | −22% |
| 25 | 173.4 | 139.6 | −33.8 | −19% |
| 26 | 240.2 | 188.6 | −51.6 | −21% |
| 27 | 273.8 | 241.2 | −32.6 | −12% |
| 28 | 265.0 | 226.0 | −39.0 | −15% |
| 29 | 237.4 | 198.2 | −39.2 | −17% |
| 30 | 248.8 | 239.6 | −9.2 | −4% |
| 31 | 162.2 | 141.0 | −21.2 | −13% |
| 32 | 183.4 | 160.8 | −22.6 | −12% |
| 33 | 189.2 | 151.0 | −38.2 | −20% |
| 34 | 172.4 | 161.0 | −11.4 | −7% |
| 35 | 180.0 | 157.0 | −23.0 | −13% |
| Average | 209.7 | 178.8 | −30.9 | −15% |

TABLE 28
SUMMARY OF RESULTS AFTER 26 WEEKS.

| 26 Week USA Summary | | |
|---|---|---|
| Participants | # | 35 |
| Trial Length | weeks | 26 |
| Average Weight | lb | −30.9 |
| Average Weight Change Per Week | lb | −1.2 |
| % Body Weight | % | −13.4 |
| Muscle Mass | lb | −7.1 |
| Fat | lb | −23.8 |
| BMI | % | −14.6 |
| Visceral Fat | % | −27.9 |
| Total Inches | inches | −23.3 |
| Waist Size | inches | −6.0 |
| Dress Size | Sizes | −3.4 |
| Pants Size | Sizes | −5.3 |
| Food Compliance | % | 73.9 |
| Exercise Compliance | % | 73.2 |
| Blood Pressure Systolic | % | −12.5 |
| Blood Pressure Diastolic | % | −5.9 |
| Total Cholesterol | % | −9.6 |
| LDL | % | −13.2% |
| HDL | % | 5.4% |
| Triglycerides | % | −31.3% |
| Glucose | % | −8.5% |
| AIC | % | −3.9% |
| Cholesterol/HDL Ratio | % | −14.2% |
| LDL/HDL Ratio | | −17.7% |
| Metabolic Syndrome Risk Factors | Baseline | 114 |
| Metabolic Syndrome Risk Factors | 26 wks. | 50 |
| Metabolic Syndrome | Baseline | 24 |
| Metabolic Syndrome | 26 wks. | 5 |
| Pre Diabetic | Baseline | 23 |
| Pre Diabetic | 26 wks. | 12 |

### Example 4

Further studies have been planned to further evidence the surprising and unexpected results shown in previous studies. Such studies may be a multi-arm study, such as two arm, three arm or four arm study for the weight loss composition, the weight management plan, and/or the bulk food product of embodiments herein. An exemplary study may include a comparison of the weight management composition of embodiments herein to a placebo, the weight management composition of embodiments herein and the weight management plan of embodiments herein to a placebo, and/or the weight management composition of embodiments herein and the weight management plan of embodiments herein to a control diet. The planned studies are expected to fully support the superior results seen in past studies.

In other embodiments, the food product can contain a decreased amount of animal protein and an increased amount of vegan protein relative to the food products described above. The vegan protein can comprise either an unprocessed plant-based protein, such as quinoa, or a processed plant-based protein, such as tofu. In these embodiments, the food product is a composition of ingredients containing protein, whether a plant-based protein(s) or an animal protein(s) in an amount ranging from about 10 wt % to about 65 wt %; a plant-based complex or starch carbohydrate in an amount ranging from 10 wt % to about 65 wt %; fats in an amount ranging from about 1 to about 5 wt %; and vegetable(s) in an amount ranging from about 20 wt % to about 50 wt %. The food products contains sufficient nutrients to meet U.S. federal dietary recommendations to promote health and reduce the risk of chronic disease.

The food product in these embodiments is designed to be consumed by an individual in a recommended serving size. This recommended serving size can vary depending on the specific ingredients in the food product. In some configurations, the recommended serving size can range from about 1% cup to about 2 cups. In other configurations, the recommended serving size can range from about ½ cups to about 1½ cups. In yet other configurations, the recommended serving size can be about 1 cup or about 6.5 oz.

The food product in these embodiments contains ingredients in the four major food groups. The first group of ingredients is protein, including both plant-based proteins and/or animal-based proteins. The second group of ingredients is plant-based complex or starch carbohydrates. The third major group of ingredients is vegetables. And the fourth major group of ingredients is fats.

The first ingredient, protein, includes both plant-based proteins and/or animal-based proteins can be added to the food product as a source of protein that also contains high fiber. In some embodiments, the amount of this ingredient that can be added to the food product can range from about 10 wt % to about 65 wt %. In other embodiments, the amount of this ingredient that can be added to the food product can range from about 15 wt % to about 50 wt %. In yet other embodiments, the amount of this ingredient that can be added to the food product can range from about 20 wt % to about 40 wt %. In even other embodiments, the amount of this ingredient can be any combination or sub-combination of these amounts. Examples of the plant-based protein(s) include beans, lentils, legumes, quinoa, nuts, tempeh, tofu, grains, and combinations thereof. There exists a wide range of beans that can be used including mung beans and/or azuki beans. Examples of the animal protein includes lean meat, such as chicken without skin, beef, pork and fish. Other examples include beef, bison, buffalo, chicken, duck, goose, hen, lamb, pork, quail, turkey, veal, venison, crab, bass, catfish, clams, cod, flounder, grouper, halibut, haddock, lobster, mussels, oysters, rockfish, salmon, scallops, shrimp, snapper, swordfish, trout, tuna, cheese (all natural), cottage cheese, cream cheese, eggs (whether alone or with bacon), milk (whole), yogurt (plain), beef broth, bone broths, and combinations thereof.

The second ingredient, plant-based complex or starch carbohydrates, can be added as a source of carbohydrates that also contains high fiber. Examples of the plant-based complex or starch carbohydrate(s) include rice, quinoa, potatoes, sweet potatoes, yams, cuscus, amaranth, barley, bread, brown rice, corn tortilla, wheat tortilla, couscous, farro, millet, spelt, oatmeal, pasta, quinoa, jicama, whole wheat, coconut flour, all varieties of beans including lentils or lima beans, edamame, hummus, wheat, and combinations thereof. In some embodiments, the amount of this ingredient that can be added to the food product can range from about 10 wt % to about 65 wt %. In other embodiments, the amount of this ingredient that can be added to the food product can range from about 15 wt % to about 50 wt %. In yet other embodiments, the amount of this ingredient that can be added to the food product can range from about 20 wt % to about 40 wt %. In even other embodiments, the amount of this ingredient can be any combination or sub-combination of these amounts.

The third major ingredient, vegetables (or a blend of vegetables), that can be added to the food product is a source of many nutrients, including fiber and vitamins. Examples of this ingredient include carrots, spinach, and other vegetables. In some embodiments, the vegetable can be a non-starchy vegetable (http://www.diabetes.org/food-and-fitness/food/what-can-i-eat/making-heal-thy-foodchoices/non-starchy-vegetables.html). In other embodiments, the vegetable source may include, but not limited to, alfalfa sprouts, artichokes, arugula, asparagus, bean sprouts, beets, bok choy, broccoli (all varieties), micro greens (all varieties), Brussels sprouts, cabbage (green), cabbage (red), carrots, cauliflower, celery, collard greens, cucumber, eggplant, fennel, green beans, kale, leeks, lettuce, mushrooms, okra, onion, parsnips, peppers (green), peppers (all other), pumpkin, radish, sauerkraut, spinach, squash (all varieties), Swiss chard, snow peas, turnips, zucchini, avocado, tomatoes, tomato sauce, olives, corn, peas, and combinations thereof. In some embodiments, the amount of this ingredient that can be added to the food product can range from about 20 wt % to about 50 wt %. In other embodiments, the amount of this third ingredient that can be added to the food product can range from about 30 wt % to about 40 wt %. In even other embodiments, the amount of this ingredient can be any combination or sub-combination of these amounts.

The fourth ingredient, fats, is added to the food product as a source of essential fats. Examples of this ingredient include butter, coconut oil, oils (olive, walnut, sesame, or avocado), homemade dressing, unsweetened coconut flakes, and combinations thereof. In some embodiments, the amount of this ingredient that can be added to the food product can range from about 1 wt % to about 5 wt %. In other embodiments, the amount of this ingredient that can be added to the food product can range from about 2 wt % to about 3 wt %. In even other embodiments, the amount of this ingredient can be any combination or sub-combination of these amounts.

The food product in these embodiments also contains two other ingredients. The first other ingredient is a sofrito mixture. And the second other ingredient is a mixture of natural preservatives and flavor enhancements (a preservative mix).

The first other ingredient, the sofrito mixture (or sofrito mix), is a mixture of oils, some vegetables, and can also include natural herbs that can be combined as a sub-blend or mixture. The sofrito mix is used for both flavor and blending purposes since the manufacturing process can create sub-blends that go into the final food product to improve the flavor and blend consistency. Thus, the sofrito mix is added to the food product as a source of both flavoring and as a natural preservative. Examples of the ingredients that could be included in the sofrito mix include vegetables, natural herbs, and spices such as sea salt, lemon, rosemary oil, rosemary extract, basil, thyme, garlic and other natural flavorings, pomegranate, pomegranate extract and combinations thereof. In other configurations, the sofrito mix comprises of a blend of garlic, red peppers, yellow onion, and avocado oil.

The sofrito mixture can be contained in the final food product in an amount up to about 50 wt %. In other configurations, the sofrito mix can contain similar ingredients in an amount up to about 40 wt % or 30 wt %. In even other configurations, the sofrito mix can contain similar ingredients in an amount up to about 10 wt % or about 20 wt %. In even other configurations, the sofrito mix can contain similar ingredients in an amount up to about 5 wt %. In some embodiments, the amount of this ingredient that can be added to the food product can range up to about 2 wt %. In even other embodiments, the amount of this ingredient can be any range, combination, or sub-combination of these amounts.

The second other ingredient, the preservative mix, is added to the food product to provide a natural preservative effect while also enhancing flavor. The preservative mix could include preservatives, herbs, enzymes, vitamins, minerals, micro-nutrients, and probiotics that can provide phytonutrients for the diet. The preservative mix can also contain a mixture/blend of these ingredients and/or some of the ingredients described herein. For example, the preservative mix could contain just herbs and oil that provide flavor and preservative effect, e.g., olive oil, rosemary extract, pomegranate, and sea salt. In some embodiments, the amount of this ingredient that can be added to the food product can range from about 0 wt % to about 5 wt %. In other embodiments, the amount of this ingredient that can be added to the food product can range from about 1 wt % to about 4 wt %. In other embodiments, the amount of this ingredient that can be added to the food product can range from about 2 wt % to about 3 wt %. In even other embodiments, the amount of this ingredient can be any combination or sub-combination of these amounts.

The above ingredients can be mixed or blended in any ratio that provide a well-balanced, plant-based complete meal with healthy and adequate grams of protein, carbohydrates, fat, fiber and nutrients. The mixture is both vegan and vegetarian compliant.

In some embodiments, the ingredients can be mixed to make the following three food products. The first composition, the vegan food composition, contains plant-based proteins. The ingredients can be mixed to make an approximate 1 cup mixture, an approximate 1.5 cup mixture, or an approximate 4.5 cup mixture as shown in Table 29A.

TABLE 29A
Vegan Food Composition

| Serving-Cups | 1 | 1.5 | 4.5 |
|---|---|---|---|
| Serving Grams | 189 | 283.5 | 850.5 |
| Quinoa | 31.0 | 46.5 | 139.6 |
| Mung Beans | 30.8 | 46.2 | 138.6 |
| Potato | 30.8 | 46.2 | 138.6 |
| Azuki Beans | 20.2 | 30.3 | 90.9 |
| Butternut | 30.8 | 46.2 | 138.6 |
| Spinach | 20.2 | 30.3 | 91.0 |
| Sofrito Mix | 19.1 | 28.7 | 86.2 |
| Preservative Mix | 6.0 | 9.0 | 27.0 |
| Total | 189 | 283.5 | 850.5 |

These same ingredients can be mixed in the same ratios to also make an approximate 1 pound mixture as shown in Table 29B, which also shows the weight percentages of each ingredient.

TABLE 29B
Vegan Food Composition

| Serving Size (1 pound) | Wt (grams) | Wt % |
|---|---|---|
| Quinoa | 74.5 | 16.41% |
| Mung Beans | 74 | 16.3% |
| Potato | 74 | 16.3% |
| Azuki Beans | 48.5 | 10.68% |
| Butternut Squash | 74 | 16.3% |
| Spinach | 48.6 | 10.7% |
| Sofrito Mix | 46 | 10.13% |
| Preservative Mix | 14.4 | 3.17% |
| Total | 454 | 100% |

The second food product, the chicken-based food composition, contains plant-based proteins and animal-based protein (chicken) and the ingredients can be mixed to make an approximate 1 cup mixture, an approximate 1.5 cup mixture, or an approximate 4.5 cup mixture as shown in Table 30A.

TABLE 30A
Chicken-Based Food Composition

| Serving-Cups | 1 | 1.5 | 4.5 |
|---|---|---|---|
| Serving Grams | 189 | 283.5 | 850.5 |
| Chicken | 41.63 | 62.4 | 187.3 |
| Quinoa | 24.8 | 37.2 | 111.7 |
| Mung Beans | 22.1 | 33.1 | 99.3 |
| Potatoes | 20.8 | 31.2 | 93.7 |
| Azuki Beans | 12.5 | 18.7 | 56.2 |
| Butternut | 21.6 | 32.5 | 97.4 |
| Spinach | 20.8 | 31.2 | 93.7 |
| Sofrito Mix | 18.7 | 28.1 | 84.3 |
| Preservative Mix | 6.0 | 9.0 | 27.0 |
| Total | 189 | 283.5 | 850.5 |

These same ingredients can be mixed in the same ratios to also make an approximate 1 pound mixture as shown in Table 30B, which also shows the weight percentages of each ingredient.

TABLE 30B
Chicken-Based Food Composition

| Serving Size (1 pound) | Wt (grams) | Wt % |
|---|---|---|
| Quinoa | 59.6 | 13.13% |
| Mung Beans | 53 | 11.67% |
| Potatoes | 50 | 11.01% |
| Azuki Beans | 30 | 6.61% |
| Butternut Squash | 52 | 11.45% |
| Spinach | 50 | 11.01% |
| Sofrito Mix | 45 | 9.91% |
| Preservative Mix | 14.4 | 3.17% |
| Chicken | 100 | 22.03% |
| Total | 454 | 100% |

The third composition, the bison-based food composition, contains plant-based proteins and animal-based protein (bison) and the ingredients can be mixed to make an approximate 1 cup mixture, an approximate 1.5 cup mixture, or an approximate 4.5 cup mixture as shown in Table 31A.

TABLE 31A
Bison-Based Food Composition

| Serving-Cups | 1 | 1.5 | 4.5 |
|---|---|---|---|
| Serving Grams | 189 | 283.5 | 850.5 |
| Bison | 41.6 | 62.4 | 187.3 |
| Quinoa | 24.8 | 37.2 | 111.7 |
| Mung Beans | 22.1 | 33.1 | 99.3 |
| Potato | 20.8 | 31.2 | 93.7 |
| Azuki Beans | 12.5 | 18.7 | 56.2 |
| Butternut | 21.6 | 32.5 | 97.4 |
| Spinach | 20.8 | 31.2 | 93.7 |
| Sofrito Mix | 18.7 | 28.1 | 84.3 |
| Preservative Mix | 6.0 | 9.0 | 27.0 |
| Total | 189 | 283.5 | 850.5 |

These same ingredients can be mixed in the same ratios to also make an approximate 1 pound mixture as shown in Table 31B, which also shows the weight percentages of each ingredient.

TABLE 31B
Bison-Based Food Composition

| Serving Size (1 pound) | Wt (grams) | Wt % |
|---|---|---|
| Quinoa | 59.6 | 13.13% |
| Mung Beans | 53 | 11.67% |
| Potatoes | 50 | 11.01% |
| Azuki Beans | 30 | 6.61% |
| Butternut Squash | 52 | 11.45% |
| Spinach | 50 | 11.01% |
| Sofrito Mix | 45 | 9.91% |
| Preservative Mix | 14.4 | 3.17% |
| Chicken | 100 | 22.03% |
| Total | 454 | 100% |

Of course, the ingredients in these 3 food products can be mixed to make other amounts, i.e., about 2 cups, about 3 cups, about 5 cups, or even more for any of these 3 food products. In these three food compositions, the sofrito mix can be a blend of red peppers (about 65 wt %), yellow onion (about 23 wt %), garlic (about 4 wt %), and avocado oil with a high heat tolerance (about 8 wt %) and the preservative mix can be a blend of olive oil (about 78 wt %), sea salt (about 7 wt %), pomegranate extract (about 14 wt %), and a rosemary extract (about 1 wt %).

The ingredients listed in Tables 29A, 29B, 30A, 30B, 31A, and 31B can be modified in other configurations of the food compositions. In some embodiments, these ingredients can be varied by up to about 5%. In other embodiments, these ingredients can be varied by up to about 10%. In other embodiments, these ingredients can be varied by up to about 20%. In even other embodiments, these ingredients can be varied by up to about 30%. In still other embodiments, these ingredients can be varied by any range, sub-range, combination, or sub-combination of these amounts.

In some embodiments, the food products can be manufactured using the following methods. The individual ingredients in the food composition except for the sofrito mix and preservative mix are all cooked separately and cooled quickly in a blast cooler/freezer to form the base ingredients when blended. The sofrito mix is cooked separately with its 4 ingredients and also cooled quickly. The preservative mix can be blended cold without using heat processing and then mixed with the sofrito mix once it is cooled. In yet other embodiments, some of the base ingredients can be mixed with the sofrito mix prior to mixing with base and also the preservative mix can be premixed into the sofrito mix. The base and sofrito/preservative mixture are combined and blended cooled in a large blender to make up to about 500 pounds, 1000 pounds, 1500 pounds, or even up to about 10,000 pounds of a bulk food product. This bulk food product can be packed into containers containing the food product with the desired serving (i.e., 6 oz, 7 oz, 8 oz, 9 oz, 10 oz, 1 cup, 1.5 cups, 3 cups, or 4.5 cups) and frozen. When served, the food products can be microwaved, stove heated, or pan heated. The refrigerator shelf life of the food products after thawing can range from about 5 to about 7 days.

In even other embodiments, the food composition can be manufactured using the following methods. The sofrito mix is made by heating the avocado oil, adding the onions and garlic and cooking until the onions are translucent, then adding the bell peppers and cooking until they are tender, and then cooling the mixture down. The preservative mix is made by mixing all of the ingredients together. The azuki beans are then cooked until they are tender and then cooled. The butternut squash is then cooked until it is tender and then pureed. The azuki beans, butternut squash, and preservative mix can then be added to the sofrito mix to create a first mixture. The quinoa, mung beans, and potato are cooked separately and cooled and then blended together to create a second mixture. Spinach can then be added to the second mixture and blended to create a third mixture. The chicken or bison are then cooked (if needed) and then added to the third mixture and blended to create a fourth mixture. The fourth mixture and the first mixture are added together and then blended to create the final food composition. The final food composition is packed into containers containing the desired serving size of the food product (i.e., 6 oz, 7 oz, 8 oz, 9 oz, 10 oz, 1 cup, 1.5 cups, 3 cups, or 4.5 cups) and then frozen. The final product could be packaged in containers with about 1, 5, 10, 15, 20, 25, or 50 lbs or even any desired size of container.

In still other embodiments, the food composition can be manufactured using the following methods. All of the ingredients are cooked separately. So the quinoa and mung beans are cooked, cooled, and blended. Then the bison or chicken is cooked and cooled. The spinach is then cooked and cooled. The butternut is also cooked and cooled. The ingredients of the sofrito mix are sautéed separately, e.g., the onion, peppers, and garlic are sauteed using avocado oil since avocado has high heat tolerance and health benefits. Separately, the ingredients of the preservative mixture (olive oil, rosemary extract, pomegranate, sea salt) are blended cold and not heated. The preservative mixture can then be added to the cooled sofrito mixture, or it could be added to the azuki beans and/or butternut squash. All of the ingredients are then blended together.

In even yet other embodiments, the food composition can be manufactured using the following methods. The mung beans, quinoa and spinach are blended together and mixed until they have a consistent blend. The potatoes are then added and blended, and then the animal protein (chicken or bison) are added, if appropriate. The sofrito mix with the preservative mix, azuki beans and butternut squash is then added as a final step and blended into the other blended ingredients.

In some configurations, the food products can be manufactured and packaged in any size desired by a consumer. In some embodiments, the food product can be manufactured and packaged with the desired serving size described above (i.e., 1 cup, 1.5 cups, 3 cups, or 4.5 cups). In other embodiments, the food product can be manufactured and packaged with a serving size of about 5, 6, 7, 8, 9, or 10 oz or multiples thereof. As well, the food product can be manufactured and packaged with any serving size useful for an individual.

In some configurations, a chosen quantity of the food composition can be prepared that is sufficient for any desired number of meals and the packaged into a container as a food product. In some embodiments, the number of servings can range from 8 to 24 meals. In other embodiments, the number of servings can range from 6 to 18 meals. In yet other embodiments, the number of servings can range from 2 or 3 to 12 meals. In still other embodiments, the number of servings in a given container can be 4 to 5 meals. The container with the food product can contain a label having a defined size with the desired amount of nutrients (i.e. 10 oz) that are recommended for consumption by the consumer. After packaging, the container and its contents can be snap frozen, refrigerated, or flash frozen. The food product can be distributed and/or sold in any convenient manner. The food product lends itself to online sales using appropriate software described herein, and then delivered to the consumer. The food product could also be delivered to retail outlets in bulk for resale by retailers or the recipe and certain portions of the food product could be provided to retailers, restaurants, etc. for preparation and sale under license. The food product can be consumed cold, can be microwaved or conventionally heated, or even mixed with liquids prior to consumption.

In other configurations, the food products described herein can be sold in bulk (i.e., as a bulk food product) rather than being sold/packaged in individual container as a food product. In these configurations, the food composition can be sold in bulk amounts of 2, 3, 4, or even 5 pounds. Individuals could use the bulk food product in many different ways. In some instances, individuals could use the bulk food product as a complete meal, i.e., digesting 1 to 2 cups depending on exact caloric intake desired by that individual. In other instances, individuals could use the bulk food product as a partial meal, i.e., digesting ½ to 1 cup depending on exact caloric intake desired by that individual, supplemented with additional foods like fruit, salad, dairy or nuts. In yet other instances, individuals could use the bulk food product as a snack, i.e., digesting ¼ to ½ cup depending on exact caloric intake desired by that individual, optionally supplemented with other foods. In still other embodiments, individuals could use the bulk food product as a hybrid meal, for example, a cup and 1 egg, 1 cup and 3-6 ounces protein (such as salmon or tofu), or 1 cup and salad, fruit, or dessert based on the caloric objective of the individual.

In some embodiments, the food product can be manufactured with a mixture of ingredients that can provide a caloric intake for a consumer depending on the weight objective of whether to lose weight, maintain weight, or gain weight. The caloric intake from the food product will therefore depend on the specific mixture of ingredients in the food product. For the 3 food compositions in Tables 29A through 31B, the caloric intake can range from about 280 to about 370 calories per 10 oz serving. The nutritional panel for each of these 3 specific food compositions are shown in Tables 32 through 34, where RDA stands for the recommended daily allowance standard set by the U.S. Food & Drug Administration.

TABLE 32
Nutritional Information for
Vegan-Based Food Composition

| Calories | | 280 | % RDA |
|---|---|---|---|
| Total Fat | g | 9.0 | 12% |
| Saturated Fat | g | 1.5 | 8% |
| Cholesterol | mg | 0.0 | 0% |
| Sodium | mg | 440.0 | 19% |
| Total Carbs | g | 42.0 | 15% |
| Fiber | g | 10 | 36% |
| Sugars | g | 4 | N/A |
| Protein | g | 13.0 | N/A |
| Vit D | mcg | 0.0 | 0% |
| Calcium | mg | 100.0 | 8% |
| Iron | mg | 3.8 | 20% |
| Potassium | mg | 890.0 | 20% |

TABLE 33
Nutritional Information for
Chicken-Based Food Composition

| Calories | | 350 | % RDA |
|---|---|---|---|
| Total Fat | g | 14 | 18% |
| Saturated Fat | g | 2.5 | 13% |
| Cholesterol | mg | 60 | 20% |
| Sodium | mg | 470.0 | 20% |
| Total Carbs | g | 32 | 12% |
| Fiber | g | 8.0 | 29% |
| Sugars | g | 3.0 | N/A |
| Protein | g | 27 | N/A |
| Vit D | mcg | 0.1 | 0% |
| Calcium | mg | 90 | 6% |
| Iron | mg | 3.2 | 20% |
| Potassium | mg | 840.0 | 20% |

TABLE 34
Nutritional Information for
Bison-Based Food Composition

| Calories | | 370 | % RDA |
|---|---|---|---|
| Total Fat | g | 19.0 | 24% |
| Saturated Fat | g | 4.0 | 20% |
| Cholesterol | mg | 25.0 | 8% |
| Sodium | mg | 530.0 | 23% |
| Total Carbs | g | 32 | 12% |
| Fiber | g | 7.0 | 25% |
| Sugars | g | 3.0 | N/A |
| Protein | g | 22 | N/A |
| Vit D | mcg | 0.0 | 0% |
| Calcium | mg | 9.0 | 6% |
| Iron | mg | 4.2 | 12% |
| Potassium | mg | 910.0 | 20% |

The caloric intake in the food compositions in these 3 tables can range from about 280 to about 370 calories for a serving of 283.5 grams. The nutritional panel for each of these 3 food compositions are shown in Tables 35A through 37A and additional nutrient information for each of these 3 specific food compositions are shown in Tables 35B through 37B.

TABLE 35B
Nutrient Information for Second Version of
Vegan-Based Food Composition

| Nutrient | Measurement | Unit | ~% DV |
|---|---|---|---|
| Excellent Source | | | ≥20% DV |
| Fiber | 9.88 | g | 35% |
| Vitamin A | 5885.07 | IU | 32% |
| Thiamin | 0.26 | mg | 21% |
| Folate | 0.26 | mcg | 49% |
| Vitamin C | 28.75 | mg | 31% |
| Vitamin K | 115.29 | mcg | 142% |
| Magnesium | 115.29 | mg | 27% |
| Copper | 0.38 | mg | 42% |
| Manganese | 1.07 | mg | 46% |
| Good source | | | 10-19% DV |
| Riboflavin | 0.21 | mg | 16% |
| Niacin | 1.83 | mg | 11% |
| Pantothenic acid | 0.66 | mg | 13% |
| Phosphorus | 232.83 | mg | 18% |
| Potassium | 882.49 | mg | 18% |
| Iron | 3.46 | mg | 19% |
| Zinc | 1.89 | mg | 17% |
| Total Vitamin E | 1.77 | mg | 11.79% |
| (alpha-tocopherol) | | | |

TABLE 36B
Nutrient Information for Second Version of
Chicken-Based Food Composition

| Nutrient | Measurement | Unit | ~% DV |
|---|---|---|---|
| Excellent Source | | | ≥20% DV |
| Fiber | 8.06 | g | 28% |
| Vitamin A | 7581.44 | IU | 42% |
| Vitamin B6 | 0.38 | mg | 22% |
| Folate | 162.6 | mcg | 40% |
| Vitamin C | 33.18 | mg | 36% |
| Vitamin K | 160.32 | mcg | 133% |
| Magnesium | 99.84 | mg | 23% |
| Copper | 0.3 | mg | 33% |
| Manganese | 0.92 | mg | 39% |
| Good source | | | 10-19% DV |
| Thiamin | 0.21 | mg | 17% |
| Riboflavin | 0.19 | mg | 14% |
| Pantothenic acid | 0.67 | mg | 13% |
| Phosphorus | 179.33 | mg | 14% |
| Potassium | 923.36 | mg | 19% |
| Iron | 3.27 | mg | 18% |
| Zinc | 1.49 | mg | 13% |
| Total Vitamin E | 1.43 | mg | 9.50% |
| (alpha-tocopherol) | | | |

TABLE 37B
Nutrient Information for Second Version
of Bison-Based Food Composition

| Nutrient | Measurement | Unit | ~% DV |
|---|---|---|---|
| Excellent Source | | | ≥220% DV |
| Fiber | 8.06 | g | 29% |
| Vitamin A | 7583.34 | IU | 42% |
| Thiamin | 0.3 | mg | 25% |
| Riboflavin | 0.35 | mg | 27% |
| Niacin | 5.17 | mg | 32% |
| Vitamin B6 | 0.64 | mg | 37% |
| Folate | 170.76 | mcg | 42% |
| Vitamin B12 | 1.32 | mcg | 55% |
| Vitamin C | 33.18 | mg | 36% |
| Vitamin K | 161.13 | mcg | 134% |
| Magnesium | 114.13 | mg | 27% |
| Selenium | 16.73 | mcg | 30% |
| Zinc | 4.62 | mg | 41% |
| Phosphorus | 311.32 | mg | 24% |
| Potassium | 994.86 | mg | 21% |
| Copper | 0.4 | mg | 43% |
| Iron | 5.08 | mg | 28% |
| Manganese | 0.92 | mg | 39% |
| Good source | | | 10-19% DV |
| Pantothenic Acid | 0.57 | mg | 11% |
| Choline | 89.41 | mg | 16% |
| Total Vitamin E | 1.8 | mg | 12% DV |
| (alpha-tocopherol) | | | |

The various ingredients of the food composition can provide phytonutrients for a diet. A phytonutrient is a substance found in certain plants and is believed to be beneficial to human health and help prevent various diseases. Phytonutrients are sometimes called phytochemicals or antioxidants. The food compositions described herein contain a wide variety of phytonutrients that offer a broad range of health benefits. Some examples of the phytonutrients and their sources include:

| Source | Phyonutrient(s) |
|---|---|
| Spinach | beta-carotene, lutein and zeaxanthin |
| Butternut squash | zeaxanthin and lutein |
| Potatoes | carotenoids, flavonoids, folates, caffeic acid |
| Mung beans | phenolic acids, flavonoids, caffeic acid, |
| | cinnamic acid |
| Azuki beans | polyphenols, anthocyanins, tannins, |
| | flavonoids, saponins |

As shown in Tables 35B, 36B, and 37B, a single serving contains many essential vitamins, minerals and fiber, as well as healthy fats and proteins that are not shown in these tables. These 3 tables are broken into both excellent and good sources of vitamins and minerals. Of course, the nutritional value as a percent of the recommended daily allowance (RDA) would significantly increase if a person consumed 2, 3, or more servings per day. Even half a serving as a snack would still be a good source of key vitamins and minerals. These amounts of vitamins, minerals and fiber and not found at these levels in un-fortified processed food.

The food compositions described herein are designed to provide maximum nutritional benefit and meet generally accepted guidelines for good nutrition, provide more fiber, use low sodium and saturated fats, all while providing maximum satiety between meals. The food compositions also contain negligible, if any, amounts of preservatives, additives, fortified products or ingredients. They also provide a better source of macro and micro nutrients than processed food, surpasses the recommended daily fiber allowances, contains no preservatives and is shelf stable for 5-7 days once thawed. The food compositions allow consumers to eat an entire balanced meal, create a balanced base meal or balanced snack to help control glucose spikes and control hunger and cravings. Consumers can use the food products described herein to lose weight, maintain weight or gain weight while improving health.

The food compositions described in these embodiments would be likely to exhibit substantially similar—if not improved—nutritional characteristics as described in Tables 5 through 28 and Examples 1 to 4 above. As well, the food products contain sufficient nutrients to meet U.S. federal dietary recommendations to promote health and reduce the risk of chronic health issues. Indeed, the food products can be part of a diet that provides a simple, serving-based pattern of eating that revolves around creating and preparing balanced meals with nutrient-dense foods, in specified serving amounts, from four of the plan's food groups: proteins, carbohydrates, vegetables, and healthy fats. Processed foods are eliminated from the diet. The number of servings can be adjusted to accommodate caloric needs that differ due to: age, gender, daily activity levels, and duration and type of exercise, as well as the individual's weight management goals. Meals are spaced apart in a 4 to 6 hour pattern between breakfast and lunch and lunch and dinner with a recommendation to fast for 12 hours between dinner and breakfast. The objective is to rest the digestive system and key organs between meals and overnight. Foods can be chosen based on individual preferences provided that serving sizes adhere to the recommendations and all four food groups are represented.

The food products can also be a part of a balanced, healthy dietary program designed to help the user to achieve a healthy body weight and in doing so promote health. Proper weight management through the dietary program encourages body fat reduction while aiming to conserve lean body mass. Reducing body weight in this balanced way and limiting weight loss to one to two pounds per week enhances plan compliance and promotes long-term success, teaching healthy eating patterns that can help maintain body weight once the desirable body weight has been achieved. These healthy eating patterns are a foundation for a positive long-term lifestyle change. The additional benefits of weight loss achieved via the dietary product and consumption of the food products described herein include: 1) an efficient metabolism that converts food into energy rather than storing it as fat; 2) replenishment of nutrients lacking in the typical diet which ensures nutritional adequacy and protects against nutrient deficiency; 3) improvements in metabolic health markers along with other health benefits associated with weight loss and improved eating patterns; and 4) reduced risk of chronic disease, including heart (cardiovascular) disease and diet-related, type 2 diabetes.

Table 38 shows the amino acid content for a single serving (283.5 g) of the second versions of the plant-based, chicken-based, and bison-based food compositions. It shows not only the essential amino acid content, but also the non-essential amino acid content.

TABLE 38
Amino Acid Content

| RDA/2.2 lbs | RDA for | PLANT | | CHICKEN | | BISON | |
|---|---|---|---|---|---|---|---|
| body | 160 lb | Per | RDA | Per | RDA | Per | RDA |
| weight (mg) | person (g) | serving (g) | % | serving (g) | % | serving (g) | % |

9 Essential Amino Acids

| | | | | | | | | |
|---|---|---|---|---|---|---|---|---|
| Histidine | 14 | 1.02 | 0.27 | 26.5% | 0.2 | 19.6% | 0.7 | 68.8% |
| Isoleucine | 19 | 1.38 | 0.42 | 30.4% | 0.31 | 22.4% | 0.98 | 70.9% |
| Leucine | 42 | 3.05 | 0.74 | 24.2% | 0.54 | 17.7% | 1.72 | 56.3% |
| Lysine | 38 | 2.76 | 0.69 | 25.0% | 0.49 | 17.7% | 1.76 | 63.7% |
| Methionine | 1. | 1.38 | 0.15 | 10.9% | 0.12 | 8.7% | 0.49 | 35.5% |
| Phenylalanine | 33 | 2.40 | 0.56 | 23.3% | 0.39 | 16.3% | 0.97 | 40.4% |
| Threonine | 20 | 1.45 | 0.38 | 26.1% | 0.26 | 17.9% | 0.89 | 61.2% |
| Trytophan | 5 | 0.36 | 0.14 | 38.5% | 0.09 | 24.8% | 0.19 | 52.3% |
| Valine | 24 | 1.75 | 0.53 | 30.4% | 0.38 | 21.8% | 1.12 | 64.2% |

Non-Essential Amino Acids

| | | | |
|---|---|---|---|
| Alanine | 0.51 | 0.35 | 1.27 |
| Arginine | 0.81 | 0.52 | 1.45 |
| Aspartic acid | 1.36 | 0.93 | 2.28 |
| Cystine | 1.76 | 0.09 | 0.25 |
| Glutamic acid | 1.76 | 1.23 | 3.47 |
| Glycine | 0.46 | 0.32 | 1.21 |
| Hydroxyproline | 0 | 0 | 0.17 |
| Proline | 0.49 | 0.34 | 1.05 |
| Serine | 0.49 | 0.34 | 0.91 |
| Tyrosine | 0.33 | 0.21 | 0.68 |

As shown in Table 38, these food compositions provide good and/or excellent levels of the 9 essential amino acids that are needed for a daily diet. While Table 38 only shows a single serving, the amounts of amino acids in the RDA for any individual could be increased by eating more servings of these food servings in a single day. It is important on a healthy diet for an individual to get a balance of amino acids throughout the day and this can be accomplished by eating a single serving of these food compositions throughout the day (e.g., at every meal). Amino acids help the body break down food into the needed nutrients, grow and repair body tissue, create hormones and brain chemicals (such as neurotransmitters), provide energy, maintain healthy skin, hair and nails, build muscle, boost the immune system, and maintain a normal digestive system.

A comparison of the nutritional value of the food compositions described here relative to another leading brand of frozen food products are shown below. The nutritional value of the other leading brands of frozen food products containing chicken are shown on the left with the nutritional value of the chicken-based food compositions described herein are shown on the right.

This comparison illustrates the benefits of the chicken-based food compositions with more plant-based protein relative to some conventional meals that use more meat. The chicken-based food compositions provide more calories, and sufficient protein levels with twice as much fiber, less cholesterol, and less sodium despite containing more healthy fat.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

1. A food composition containing plant-based foods, comprising:
protein selected from a plant-based protein and/or animal-based protein in an amount ranging from about 10 wt % to about 65 wt %;
plant-based complex or starch carbohydrate in an amount ranging from about 10 wt % to about 65 wt %;
vegetables in an amount ranging from about 20 wt % to about 50 wt %;
fats in an amount ranging from about 1 to about 5 wt %;
natural herbs in an amount ranging up to about 2 wt %; and
a preservative mixture in an amount ranging up to about 5 wt %.

2. The food composition of claim 1, wherein the plant-based complex or starch carbohydrate is selected from rice, quinoa, potatoes, sweet potatoes, yams, cuscus, amaranth, barley, bread, brown rice, corn tortilla, wheat tortilla, couscous, farro, millet, spelt, oatmeal, pasta, quinoa, jicama, whole wheat, coconut flour, all varieties of beans including lentils or lima beans, edamame, hummus, wheat, and combinations thereof.

3. The food composition of claim 1, wherein the plant-based protein is selected from beans, lentils, legumes, quinoa, nuts, temph, tofu, grains, and combinations thereof.

4. The food composition of claim 1, wherein the animal-based protein is selected from chicken without skin, beef, pork and fish, beef, bison, buffalo, chicken, duck, goose, hen, lamb, pork, quail, turkey, veal, venison, crab, bass, catfish, clams, cod, flounder, grouper, halibut, haddock, lobster, mussels, oysters, rockfish, salmon, scallops, shrimp, snapper, swordfish, trout, tuna, beef broth, bone broths, and combinations thereof.

5. The food composition of claim 1, wherein the vegetables are selected from butternut squash, alfalfa sprouts, artichokes, arugula, asparagus, bean sprouts, beets, bok choy, broccoli, micro greens, Brussels sprouts, cabbage, carrots, cauliflower, celery, collard greens, cucumber, eggplant, fennel, green beans, kale, leeks, lettuce, mushrooms, okra, onion, parsnips, peppers, pumpkin, radish, sauerkraut, spinach, squash Swiss chard, snow peas, turnips, zucchini, avocado, tomatoes, tomato sauce, olives, corn, peas, and combinations thereof.

6. The food composition of claim 1, further comprising a sofrito mix with a blend of red peppers, garlic, yellow onion, and avocado oil with a high heat tolerance.

7. The food composition of claim 1, wherein the preservative mix comprises olive oil, sea salt, pomegranate extract, and a rosemary extract.

8. The food composition of claim 1, wherein the plant-based complex or starch carbohydrate is selected from quinoa, potatoes, sweet potatoes, and combinations thereof.

9. The food composition of claim 1, wherein the plant-based protein is selected from mung beans, azuki beans, quinoa, and combinations thereof.

10. The food composition of claim 1, wherein the animal-based protein is selected from chicken or bison.

11. The food composition of claim 1, wherein the vegetables are selected from butternut squash, spinach, and combinations thereof.

12. The food composition of claim 1, comprising quinoa, mung beans, potato, azuki beans, butternut squash, spinach, red peppers, yellow onion, avocado oil, olive oil, sea salt, pomegranate extract, and rosemary extract.

13. The food composition of claim 12, further comprising chicken or bison.

14. A food composition containing plant-based foods, comprising:
protein selected from a plant-based protein and/or animal-based protein in an amount ranging from about 10 wt % to about 65 wt %, the plant-based protein selected from mung beans and azuki beans;
plant-based complex or starch carbohydrate in an amount ranging from about 10 wt % to about 65 wt %, the plant-based complex or starch carbohydrate selected from quinoa, potatoes, and sweet potatoes;
vegetables selected from butternut squash and spinach in an amount ranging from about 20 wt % to about 50 wt %;
a sofrito mix comprising a blend of garlic, red peppers, yellow onion, and avocado oil in an amount up to about 20 wt %; and
a preservative mix of olive oil, sea salt, pomegranate extract, and a rosemary extract in an amount up to about 5 wt %.

15. The food composition of claim 14, wherein the animal protein is selected from chicken or bison.

16. A method for managing weight, comprising instructing a consumer to consume the food composition of claim 14.

17. A method for managing weight, comprising administering to a consumer the food composition of claim 14.

18. A food composition, comprising:
about 16 wt % quinoa;
about 16 wt % mung beans;
about 16 wt % potatoes;
about 11 wt % azuki beans;
about 16 wt % butternut squash;
about 11 wt % spinach;
about 10 wt % of a sofrito mix comprising garlic, red peppers, yellow onion, and avocado oil; and
about 3 wt % of a preservative mix of olive oil, sea salt, pomegranate extract, and a rosemary extract.

19. The food composition of claim 18, wherein the sofrito mix contains about 8 wt % avocado oil, about 23 wt % yellow onion, about 4 wt % garlic, and about 65 wt % red pepper.

20. The food composition of claim 18, wherein the preservative mix contains about 78 wt % olive oil, about 7 wt % sea salt, about 14 wt % pomegranate extract, and about 1 wt % rosemary extract.

21. A food composition, comprising:
about 22 wt % chicken or bison;
about 13 wt % quinoa;
about 12 wt % mung beans;
about 11 wt % potatoes;
about 7 wt % azuki beans;
about 11 wt % butternut squash;
about 11 wt % spinach;
about 10 wt % of a sofrito mix comprising garlic, red peppers, yellow onion, and avocado oil; and
about 3 wt % of a preservative mix of olive oil, sea salt, pomegranate extract, and a rosemary extract.

22. The food composition of claim 18, wherein the sofrito mix contains about 8 wt % avocado oil, about 23 wt % yellow onion, about 4 wt % garlic, and about 65 wt % red pepper.

23. The food composition of claim 18, wherein the preservative mix contains about 78 wt % olive oil, about 7 wt % sea salt, about 14 wt % pomegranate extract, and about 1 wt % rosemary extract.
